(19) United States
(12) Patent Application Publication
(10) Pub. No.: US 2021/0145739 A1
(43) Pub. Date: May 20, 2021

(54) ANTIFUNGAL FORMULATIONS FOR PULMONARY ADMINISTRATION COMPRISING ITRACONAZOLE

(71) Applicant: PULMATRIX OPERATING COMPANY, INC., Lexington, MA (US)

(72) Inventors: Jason M PERRY, Cambridge, MA (US); David L HAVA, Natick, MA (US); Robert C SAUNDERS, Arlington, MA (US); Andrew E O'CONNOR, Somerville, MA (US); Dayna HANSON, Natick, MA (US)

(21) Appl. No.: 17/048,491

(22) PCT Filed: Apr. 18, 2019
(86) PCT No.: PCT/US2019/028089

§ 371 Date: Oct. 16, 2020

Related U.S. Application Data
(60) Provisional application No. 62/659,479, filed on Apr. 18, 2018.

(51) Int. Cl. A61K 9/00; A61M 15/00; A61K 9/14; A61K 31/496

(52) U.S. Cl. CPC A61K 9/0075; A61K 31/496; A61K 9/145; A61M 15/003

(57) ABSTRACT

The invention relates to dry powder formulations comprising respirable dry particles that contain 1) itraconazole in crystalline particulate form, 2) polysorbate 80, and 3) one or more excipients, wherein the ratio of itraconazole to polysorbate 80 (wt:wt) in the feedstock solution is greater than 10:1.

This application claims the benefit of U.S. Patent Application No. 62/659,479, filed on Apr. 18, 2018, the entire contents of which are incorporated herein by reference.

### BACKGROUND

Pulmonary fungal infections by Aspergillus spp. and other fungi are a growing concern in patients with decreased respiratory function, such as cystic fibrosis (CF) patients. For example, patients can have chronic pulmonary fungal infection or Allergic Bronchopulmonary Aspergillosis (ABPA), a severe inflammatory condition that is typically treated with a long course of oral steroids. A number of antifungal agents are known including triazoles (e.g., itraconazole), polyenes (e.g., amphotericin B), and echinocandins. Antifungal agents typically have low aqueous solubility and poor oral bioavailability and obtaining pharmaceutical formulations that provide safe and therapeutic levels of antifungal agents has been challenging. Antifungal agents are typically administered as oral or intravenous (IV) formulations as treatments for fungal infections, including pulmonary infection and ABPA. However, such formulations are limited by poor oral bioavailability, adverse side effects and toxicity, and extensive drug-drug interactions. Alternative approaches, such as delivery to the airway by inhalation, which theoretically could reduce systemic side effects also present challenges. Notably, it is well known that agents with poor aqueous solubility produce local lung toxicity (e.g., local inflammation, granuloma) when inhaled. The conventional approach to address local toxicity of poorly soluble agents is to formulate the agent to increase its rate of dissolution, for example using amorphous formulations.

The chemical structure of itraconazole is described in U.S. Pat. No. 4,916,134. Itraconazole is a triazole antifungal agent providing therapeutic benefits (e.g., in the treatment of fungal infections), and is the active ingredient in SPORANOX® (itraconazole; Janssen Pharmaceuticals) which may be delivered orally or intravenously. Itraconazole can be synthesized using a variety of methods that are well known in the art.

A need exists for new formulations of antifungal agents that can safely be administered to treat fungal infections.

### SUMMARY OF THE INVENTION

The invention relates to dry powders comprising homogenous respirable dry particles that comprise 1) itraconazole in crystalline particulate form, 2) polysorbate 80, and 3) one or more excipients, wherein the ratio of itraconazole to polysorbate 80 (wt:wt) in the feedstock solution is greater than 10:1, with the proviso that the dry powder formulation does not comprise: 20% Itraconazole, 39% sodium sulfate, 39% mannitol, and 2% polysorbate 80; 50% Itraconazole, 22.5% sodium sulfate, 22.5% mannitol, and 5% polysorbate 80; 20% Itraconazole, 62.4% sodium chloride, 15.6% leucine, and 2% polysorbate 80; 50% Itraconazole, 36% sodium sulfate, 9% leucine, and 5% polysorbate 80; 20% Itraconazole, 66.3% magnesium lactate, 11.7% leucine, and 2% polysorbate 80; 50% Itraconazole, 38.25% magnesium lactate, 6.75% leucine, and 5% polysorbate 80; 50% Itraconazole, 35% sodium sulfate, 10% leucine, and 5% polysorbate 80; 50% Itraconazole, 35% sodium sulfate, 10% leucine, and less than 5% polysorbate 80; 50% Itraconazole, 35% sodium sulfate, 13.75% leucine, and 1.25% polysorbate 80; 50% Itraconazole, 37% sodium sulfate, 8% leucine, and 5% polysorbate 80; 60% Itraconazole, 26% sodium sulfate, 8% leucine, and 6% polysorbate 80; 70% Itraconazole, 15% sodium, 8% leucine, and 7% polysorbate 80; 75% Itraconazole, 9.5% sodium sulfate, 8% leucine, and 7.5% polysorbate 80; 80% Itraconazole, 4% sodium sulfate, 8% leucine, and 8% polysorbate 80; 80% Itraconazole, 10% sodium sulfate, 2% leucine, and 8% polysorbate 80; 80% Itraconazole, 11% sodium sulfate, 1% leucine, and 8% polysorbate 80; or 80% Itraconazole, 11% sodium sulfate, 1% leucine, and 8% polysorbate 80.

The sub-particle may be about 50 nm to about 5,000 nm (Dv50), about 50 nm to about 800 nm (Dv50), about 50 nm to about 300 nm (Dv50), about 50 nm to about 200 nm (Dv50), about 100 nm to about 300 nm (Dv50).

The sub-particle may be about 50 nm to about 2,500 nm (Dv50) or about 80 nm to about 1,750 nm (Dv50).

The itraconazole may be present in an amount of about 1% to about 95% by weight, about 40% to about 90% by weight, about 55% to about 85% by weight, about 55% to about 75% by weight, about 65% to about 85% by weight, about 40% to about 60% by weight. The itraconazole can at least 50% crystalline.

The ratio of itraconazole:polysorbate 80 (wt:wt) may be from about 11.5:1 to 14:1, greater than or equal to 12:1 or about 12:1, or about 15:1 to about 19.5:1.

The polysorbate 80 may be present in an amount of about 0.05% to about 45% by weight, about 4% to about 10% by weight.

The one or more excipients may be present in an amount of about 3% to about 99% by weight, or about 5% to about 50% by weight. The excipient may be a sodium salt. The one or more excipients may comprise a monovalent metal cation salt, a divalent metal cation salt, an amino acid, a sugar alcohol, or combinations thereof. The one or more excipients may comprise a sodium salt and an amino acid. The sodium salt may be selected from the group consisting of sodium chloride and sodium sulfate, and the amino acid is leucine. The sodium salt may be sodium chloride and the amino acid may be leucine. The sodium salt may be sodium sulfate and the amino acid may be leucine.

The one or more excipients may comprise a magnesium salt and an amino acid. The magnesium salt may be magnesium lactate, and the amino acid may be leucine.

The polysorbate 80 may be present in an amount of less than 10 wt %, less than 7 wt %, and less than 3 wt %.

The respirable dry particles may have a volume median geometric diameter (VMGD) about 10 microns or less, or about 5 microns or less.

The respirable dry particles may have a tap density of about 0.2 g/cc or greater or a tap density of between 0.2 g/cc and 1.0 g/cc.

The respirable dry particles may have a tap density of greater than about 0.4 g/cc to about 1.2 g/cc.

The dry powder may have an MMAD of between about 1 micron and about 5 microns.

The dry particles may have a 1 bar/4 bar dispersibility ratio (1/4 bar) of less than about 1.5 as measured by laser diffraction.

The dry particles may have a 0.5 bar/4 bar dispersibility ratio (0.5/4 bar) of about 1.5 or less as measured by laser diffraction.

The dry powder may have a FPF of the total dose less than 5 microns of about 25% or more.

The dry powder may be delivered to a patient with a capsule-based passive dry powder inhaler.

The respirable dry particles may have a capsule emitted powder mass of at least 80% when emitted from a passive dry powder inhaler that has a resistance of about 0.036 sqrt(kPa)/liters per minute under the following conditions; an inhalation flow rate of 30 LPM for a period of 3 seconds using a size 3 capsule that contains a total mass of 10 mg, said total mass consisting of the respirable dry particles, and wherein the volume median geometric diameter of the respirable dry particles emitted from the inhaler as measured by laser diffraction is 5 microns or less.

In one aspect, the invention relates to a liquid formulation that comprises 1) itraconazole in crystalline particulate form, 2) polysorbate 80, and 3) one or more excipients, wherein the ratio of itraconazole to polysorbate 80 (wt:wt) in the formulation is greater than 10:1. The itraconazole crystalline particulate form may be suspended in a propellant selected from the group consisting of HFA propellant and CFC propellant. The liquid formulation may further comprise a surfactant.

In one aspect the invention relates to a method for treating a fungal infection comprising administering to the respiratory tract of a patient in need thereof an effective amount of a dry powder or a liquid formulation described herein.

In one aspect the invention relates to a method for treating a fungal infection in a patient with cystic fibrosis comprising administering to the respiratory tract of the cystic fibrosis patient an effective amount of a dry powder or a liquid formulation described herein.

In one aspect, the invention relates to a method for treating a fungal infection in a patient with asthma comprising administering to the respiratory tract of the asthma patient an effective amount of a dry powder or a liquid formulation described herein.

In one aspect, the invention relates to a method for treating aspergillosis comprising administering to the respiratory tract of a patient in need thereof an effective amount of a dry powder or a liquid formulation described herein.

In one aspect, the invention relates to a method for treating allergic bronchopulmonary aspergillosis (ABPA) comprising administering to the respiratory tract a patient in need thereof an effective amount of a dry powder or a liquid formulation described herein.

In one aspect, the invention relates to a method for treating or reducing the incidence or severity of an acute exacerbation of a respiratory disease comprising administering to the respiratory tract of a patient in need thereof an effective amount of a dry powder or a liquid formulation, wherein the acute exacerbation is a fungal infection.

In one aspect, the invention relates to a method for treating a fungal infection in an immunocompromised patient comprising administering to the respiratory tract of the immunocompromised patient an effective amount of a dry powder or a liquid formulation described herein.

In one aspect, the invention relates to a dry powder or liquid formulation for use in treating a fungal infection in an individual, the use comprising administering to the respiratory tract of the individual an effective amount of the dry powder, wherein the fungal infection is treated.

In one aspect, the invention relates to a dry powder or liquid formulation for use in treating a fungal infection in a cystic fibrosis patient, the use comprising administering to the respiratory tract of the individual an effective amount of the dry powder, wherein the fungal infection in the cystic fibrosis patient is treated.

In one aspect, the invention relates to a dry powder or a liquid formulation for use in treating a fungal infection in an asthma patient, the use comprising administering to the respiratory tract of the individual an effective amount of the dry powder, wherein the fungal infection in the asthma patient is treated.

In one aspect, the invention relates to a dry powder or a liquid formulation for use in treating aspergillosis in an individual, the use comprising administering to the respiratory tract of the individual an effective amount of the dry powder, wherein the aspergillosis is treated.

In one aspect, the invention relates to a dry powder or a liquid formulation for use in treating allergic bronchopulmonary aspergillosis (ABPA) in an individual, the use comprising administering to the respiratory tract of the individual an effective amount of the dry powder, wherein the allergic bronchopulmonary aspergillosis (ABPA) is treated.

In one aspect, the invention relates to a dry powder or a liquid formulation for use in treating an acute exacerbation of a respiratory disease in an individual, the use comprising administering to the respiratory tract of the individual an effective amount of the dry powder, wherein the acute exacerbation is treated.

In one aspect, the invention relates to a dry powder or a liquid formulation for use in treating a fungal infection in an immunocompromised patient, the use comprising administering to the respiratory tract of the immunocompromised patient an effective amount of the dry powder, wherein the fungal infection is treated.

In one aspect, the invention relates to a dry powder or a liquid formulation produced by a process comprising the steps of: spray drying a surfactant-stabilized suspension with optional excipients, wherein dry particles that are compositionally homogeneous are produced.

### BRIEF DESCRIPTION OF THE DRAWINGS

FIG. 1 is a graph showing a comparison of the aerosol performance of the 12:1 itraconazole:PS80 formulations to the 10:1 itraconazole:PS80 formulations.

### DETAILED DESCRIPTION OF THE INVENTION

This disclosure relates to superior respirable dry powders that contain itraconazole in crystalline particulate form. The inventors have discovered that specific dry powder formulations that contain itraconazole in amorphous form have shorter lung residence times, reduced lung to plasma exposure ratios and undesirable toxic effects on lung tissue when inhaled at therapeutic doses. Without wishing to be bound by any particular theory, it is believed that the crystalline forms (e.g., nanocrystal line forms) of itraconazole have a slower dissolution rate in the lung, providing more continuous exposure over a 24 hour period after administration and minimizing systemic exposure. In addition, the observed local toxicity in lung tissue with amorphous dosing is not related to the total exposure of the lung tissue to the drug, in terms of total dose or duration of exposure. Itraconazole has no known activity against human or animal lung cells and so increasing local concentration has no local pharmacological activity to explain the local toxicity. Instead, the toxicity of the amorphous form appears related to the increased solubility of the amorphous form of the itraconazole, resulting in supersaturation of the drug in the interstitial space and recrystallization in the tissue leading to local, granulomatous inflammation. Surprisingly, the inventors discovered that dry powders that contain itraconazole in crystalline particulate form are less toxic to lung tissue. This was surprising because the itraconazole in crystalline particulate form has a lower aqueous solubility in comparison to the amorphous form and remains in the lung longer than a corresponding dose of itraconazole in amorphous form.

The crystallinity of the itraconazole, as well as the size of the itraconazole crystalline particles, appears to be important for effective therapy and for reduced toxicity in the lung. Without wishing to be bound by any particular theory, it is believed that crystalline particles of itraconazole (e.g., nano-crystalline or micro-crystalline antifungal agent) will dissolve in the airway lining fluid more rapidly than larger crystalline particles—in part due to the larger total amount of surface area. It is also believed that crystalline itraconazole will dissolve more slowly in the airway lining fluid than the amorphous itraconazole—in part due to the lower aqueous solubility. Accordingly, the dry powders described herein can be formulated using itraconazole in crystalline particulate form that provides for itraconazole in a desired crystalline size or range of crystalline sizes within the dry powders, and can be tailored to achieve desired pharmacokinetic properties while avoiding unacceptable toxicity in the lungs.

The respirable dry powders of this invention have increased ratios of itraconazole to polysorbate 80, which show surprising improvements in aerosol delivery and performance. While previously described respirable dry powders comprising 1) itraconazole in crystalline particulate form, 2) polysorbate 80, and optionally 3) one or more excipients are known to have desirable aerosol characteristics and dispersibility, as evidenced from high delivered fine-particle dose and a low 1:4 bar or 0.5:4 bar VMGD quotient, the respirable dry powders of the present invention demonstrate an increased ability to emit from a dry powder inhaler under similar flow conditions. Without wishing to be bound by any particular theory, it is believed that polysorbate 80, which is a liquid at room temperature and atmospheric pressure, increases the adhesive and cohesive forces of the particles and/or the propensity of the particles to absorb environmental moisture, as it is known to be hygroscopic. Thus, the inventors have made a surprising and unexpected discovery that the amount of PS80 in the formulation is responsible for a measurable change in performance.

The respirable dry powders of this disclosure include homogenous respirable dry particles that contain 1) itraconazole in crystalline particulate form, 2) polysorbate 80, and optionally 3) one or more excipients, wherein the ratio of itraconazole to polysorbate 80 (w/w) is greater than 10:1, about 12:1, greater than 10:1 to 15:1, preferably greater than 10:1 to 25:1, more preferably 11:1 to 15:1. Accordingly, the dry powders are characterized by respirable dry particles that contain polysorbate 80, optionally one or more excipients, and one or more sub-particles (i.e., particles that are smaller than the respirable dry particle) that comprise crystalline itraconazole. Such respirable dry particles can be prepared using any suitable method, such as by preparing a feedstock in which itraconazole in crystalline particulate form is suspended in an aqueous solution of excipients, and spray drying the feedstock. Such respirable dry particles can be prepared using any suitable method, such as by preparing a nanoparticle suspension of itraconazole in crystalline particulate form suspended in an aqueous solution which contains polysorbate 80 in sufficient amounts to stabilize the suspension. The stabilized nanoparticle suspension can then be added to another solvent (either water or another solvent which is miscible with water and in which, like water, the nanoparticles of crystalline itraconazole are poorly soluble) in which the suspension is maintained and one or more excipients is solubilized making the feedstock. This feedstock can then be spray dried to form the respirable dry particles.

The respirable dry powders of this disclosure include homogenous respirable dry particles having formulations that increase the ratio of crystalline itraconazole to polysorbate 80 to be greater than 10:1, greater than 10:1 to 25:1, 11:1 to 35:1, 10.5:1 to 14.5:1, 11:1 to 31:1, 11:1 to 15:1, 11.5:1 to 14:1, 13:1 to 16:1, 15:1 to 19.5:1, 19:1 to 25:1, 20.5:1 to 23:1, 22:1 to 32:1.

The dry powders may be administered to a patient by inhalation, such as oral inhalation. To achieve oral inhalation, a dry powder inhaler may be used, such as a passive dry powder inhaler. The dry powder formulations can be used to treat or prevent fungal infections in a patient, such as aspergillus infections. Patients that would benefit from the dry powders are, for example, those who suffer from cystic fibrosis, asthma, and/or who are at high risk of developing fungal infections due to being severely immunocompromised. An inhaled formulation of itraconazole minimizes many of the downsides of oral or intravenous (IV) formulations in treating these patients.

### Definitions

As used herein, the term “about” refers to a relative range of plus or minus 5% of a stated value, e.g., “about 20 mg” would be 20 mg plus or minus 1 mg.

As used herein, the terms “administration” or “administering” of respirable dry particles refers to introducing respirable dry particles to the respiratory tract of a subject.

As used herein, the term “amorphous” indicates lack of significant crystallinity when analyzed via powder X-ray diffraction (XRD).

The term “capsule emitted powder mass” or “CEPM” as used herein refers to the amount of dry powder formulation emitted from a capsule or dose unit container during actuation from the dry powder inhaler, such as during an inhalation maneuver. CEPM is measured gravimetrically, typically by weighing a capsule before and after the emission event to determine the mass of powder removed. CEPM can be expressed either as the mass of powder removed, in milligrams, or as a percentage of the initial filled powder mass in the capsule prior to the emission event.

The term “crystalline particulate form” as used herein refers to itraconazole (including pharmaceutically acceptable forms thereof including salts, hydrates, enantiomers as the like), that is in the form of a particle (i.e., sub-particle that is smaller than the respirable dry particles that comprise the dry powders disclosed herein) and in which the itraconazole is at least about 50% crystalline. The percent crystallinity of itraconazole refers to the percentage of the compound that is in crystalline form relative to the total amount of compound present in the sub-particle. If desired, the antifungal agent can be at least about 60%, at least about 70%, at least about 80%, at least about 90%, at least about 95%, or about 100% crystalline. Itraconazole in crystalline particulate form is in the form of a particle that is about 50 nanometers (nm) to about 5,000 nm volume median diameter (Dv50), preferably 80 nm to 1750 nm Dv50, or preferably 50 nm to 800 nm Dv50.

The term “dispersible” is a term of art that describes the characteristic of a dry powder or respirable dry particles to be dispelled into a respirable aerosol. Dispersibility of a dry powder or respirable dry particles is expressed herein, in one aspect, as the quotient of the volumetric median geometric diameter (VMGD) measured at a dispersion (i.e., regulator) pressure of 1 bar divided by the VMGD measured at a dispersion (i.e., regulator) pressure of 4 bar, or VMGD at 0.5 bar divided by the VMGD at 4 bar as measured by laser diffraction, such as with a HELOS/RODOS. These quotients are referred to herein as “1 bar/4 bar dispersibility ratio” and “0.5 bar/4 bar dispersibility ratio”, respectively, and dispersibility correlates with a low quotient. For example, 1 bar/4 bar dispersibility ratio refers to the VMGD of a dry powder or respirable dry particles emitted from the orifice of a RODOS dry powder disperser (or equivalent technique) at about 1 bar, as measured by a HELOS or other laser diffraction system, divided by the VMGD of the same dry powder or respirable dry particles measured at 4 bar by HELOS/RODOS. Thus, a highly dispersible dry powder or respirable dry particles will have a 1 bar/4 bar dispersibility ratio or 0.5 bar/4 bar dispersibility ratio that is close to 1.0. Highly dispersible powders have a low tendency to agglomerate, aggregate or clump together and/or, if agglomerated, aggregated or clumped together, are easily dispersed or de-agglomerated as they emit from an inhaler and are breathed in by a subject. In another aspect, dispersibility is assessed by measuring the particle size emitted from an inhaler as a function of flowrate. As the flow rate through the inhaler decreases, the amount of energy available to disperse the powder decreases. A highly dispersible powder will have a size distribution such as is characterized aerodynamically by its mass median aerodynamic diameter (MMAD) or geometrically by its VMGD that does not substantially increase over a range of flow rates typical of inhalation by humans, such as about 15 to about 60 liters per minute (LPM), about 20 to about 60 LPM, or about 30 LPM to about 60 LPM. A highly dispersible powder will also have an emitted powder mass or dose, or a capsule emitted powder mass or dose, of about 80% or greater even at the lower inhalation flow rates. VMGD may also be called the volume median diameter (VMD), x50, or Dv50.

The term “dry particles” as used herein refers to respirable particles that may contain up to about 15% total of water and/or another solvent. Preferably, the dry particles contain water and/or another solvent up to about 10% total, up to about 5% total, up to about 1% total, or between 0.01% and 1% total, by weight of the dry particles, or can be substantially free of water and/or other solvent.

The term “dry powder” as used herein refers to compositions that comprise respirable dry particles. A dry powder may contain up to about 15% total of water and/or another solvent. Preferably the dry powder contain water and/or another solvent up to about 10% total, up to about 5% total, up to about 1% total, or between 0.01% and 1% total, by weight of the dry powder, or can be substantially free of water and/or other solvent. In one aspect, the dry powder is a respirable dry powder.

The term “effective amount,” as used herein, refers to the amount of agent needed to achieve the desired effect; such as treating a fungal infection, e.g., an aspergillus infection, in the respiratory tract of a patient, e.g., a cystic fibrosis (CF) patient, an asthma patient and an immunocompromised patient; treating allergic bronchopulmonary aspergillosis (ABPA); and treating or reducing the incidence or severity of an acute exacerbation of a respiratory disease. The actual effective amount for a particular use can vary according to the particular dry powder or respirable dry particle, the mode of administration, and the age, weight, general health of the subject, and severity of the symptoms or condition being treated. Suitable amounts of dry powders and dry particles to be administered, and dosage schedules for a particular patient can be determined by a clinician of ordinary skill based on these and other considerations.

As used herein, the term “emitted dose” or “ED” refers to an indication of the delivery of a drug formulation from a suitable inhaler device after a firing or dispersion event. More specifically, for dry powder formulations, the ED is a measure of the percentage of powder that is drawn out of a unit dose package and that exits the mouthpiece of an inhaler device. The ED is defined as the ratio of the drug or powder delivered by an inhaler device to the nominal dose (i.e., the mass of drug or powder per unit dose placed into a suitable inhaler device prior to firing). The ED is an experimentally-measured parameter, and can be determined using the method of USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, Md., 13th Revision, 222-225, 2007. This method utilizes an in vitro device set up to mimic patient dosing. It can also be calculated from the results generated by Next Generation Impactor (NGI) experiments, through summation of all of the drug or powder assayed from the mouthpiece adapter, NGI induction port, and all of the stages within the NGI. The results generated through ED testing per USP 601 and the results generated via the NGI are typically in good agreement.

The term “nominal dose” as used herein refers to an individual dose of itraconazole. The nominal dose is the total dose of itraconazole within one capsule, blister, or ampule.

The terms “FPF (<X),” “FPF (<X microns),” and “fine particle fraction of less than X microns” as used herein, wherein X equals, for example, 3.4 microns, 4.4 microns, 5.0 microns or 5.6 microns, refer to the fraction of a sample of dry particles that have an aerodynamic diameter of less than X microns. For example, FPF (<X) can be determined by dividing the mass of respirable dry particles deposited on stage two and on the final collection filter of a two-stage collapsed Andersen Cascade Impactor (ACI) by the mass of respirable dry particles weighed into a capsule for delivery to the instrument. This parameter may also be identified as “FPF_TD(<X),” where TD means total dose. A similar measurement can be conducted using an eight-stage ACI. An eight-stage ACI cutoffs are different at the standard 60 L/min flowrate, but the FPF_TD(<X) can be extrapolated from the eight-stage complete data set. The eight-stage ACI result can also be calculated by the USP method of using the dose collected in the ACI instead of what was in the capsule to determine FPF. Similarly, a seven-stage Next Generation Impactor (NGI) can be used.

The terms “FPD (<X)”, ‘FPD<X microns”, FPD(<X microns)” and “fine particle dose of less than X microns” as used herein, wherein X equals, for example, 3.4 microns, 4.4 microns, 5.0 microns or 5.6 microns, refer to the mass of a therapeutic agent delivered by respirable dry particles that have an aerodynamic diameter of less than X micrometers. FPD<X microns can be determined by using an eight-stage ACI at the standard 60 L/min flowrate and summing the mass deposited on the final collection filter, and either directly calculating or extrapolating the FPD value. Similarly, a seven-stage Next Generation Impactor (NGI) can be used.

The term “respirable” as used herein refers to dry particles or dry powders that are suitable for delivery to the respiratory tract (e.g., pulmonary delivery) in a subject by inhalation. Respirable dry powders or dry particles have a mass median aerodynamic diameter (MMAD) of less than about 10 microns, preferably about 5 microns or less.

As used herein, the term “respiratory tract” includes the upper respiratory tract (e.g., nasal passages, nasal cavity, throat, pharynx and larynx), respiratory airways (e.g., trachea, bronchi, and bronchioles) and lungs (e.g., respiratory bronchioles, alveolar ducts, alveolar sacs, and alveoli).

As used herein, the term “lower respiratory tract” includes the respiratory airways (e.g., trachea, bronchi, and bronchioles) and lungs (e.g., respiratory bronchioles, alveolar ducts, alveolar sacs, and alveoli).

The term “small” as used herein to describe respirable dry particles refers to particles that have a volume median geometric diameter (VMGD) of about 10 microns or less, preferably about 5 microns or less, or less than 5 microns.

The term “stabilizer” as used herein refers to a compound that improves the physical stability of itraconazole in crystalline particulate form when suspended in a liquid in which the itraconazole is poorly soluble (e.g., reduces the aggregation, agglomeration, Ostwald ripening and/or flocculation of the particulates). Suitable stabilizers are surfactants and amphiphilic materials and include Polysorbates (PS; polyoxyethylated sorbitan fatty acid esters), such as PS20, PS40, PS60 and PS80; fatty acids such as lauric acid, palmitic acid, myristic acid, oleic acid and stearic acid; sorbitan fatty acid esters, such as Span20, Span40, Span60, Span80, and Span 85; phospholipids such as dipalmitoylphosphosphatidylcholine (DPPC), 1,2-Dipalmitoyl-sn-glycero-3-phospho-L-serine (DPPS), 1,2-Dipalmitoyl-sn-glycero-3-phosphocholine (DSPC), 1-palmitoyl-2-oleoylphosphatidylcholine (POPC), and 1,2-Dioleoyl-sn-glycero-3-phosphocholine (DOPC); Phosphatidylglycerols (PGs) such as diphosphatidyl glycerol (DPPG), DSPG, DPPG, POPG, etc.; 1,2-Distearoyl-sn-glycero-3-phosphoethanolamine (DSPE); fatty alcohols; benzyl alcohol, polyoxyethylene-9-lauryl ether; glycocholate; surfactin; poloxomers; polyvinylpyrrolidone (PVP); PEG/PPG block co-polymers (Pluronics/Poloxamers); polyoxyethyene chloresteryl ethers; POE alky ethers; tyloxapol; lecithin; and the like. Preferred stabilizers are polysorbates and fatty acids. A particularly preferred stabilizer is PS80. Another preferred stabilizer is oleic acid.

The term “homogenous dry particle” as used herein refers to particles containing crystalline drug (e.g., nano-crystalline drug) which is pre-processed as a surfactant stabilized suspension. The homogenous dry particle is then formed by spray drying the surfactant-stabilized suspension with (optional) excipients, resulting in dry particles that are compositionally homogenous, or more specifically, identical in their composition of surfactant-coated crystalline drug particles and optionally one or more excipients.

### Dry Powders and Dry Particles

The invention relates to dry powder formulations comprising respirable dry particles that contain 1) itraconazole in crystalline particulate form, 2) polysorbate 80, and 3) one or more excipients, wherein the ratio of crystalline itraconazole to polysorbate 80 is greater than about 10:1, greater than 10:1 to 25:1, 11:1 to 35:1, 10.5:1 to 14.5:1, 11:1 to 31:1, greater than 12:1, 11:1 to 15:1, 11.5:1 to 14:1, 13:1 to 16:1, or 15:1 to 19.5:1, 19:1 to 25:1, 20.5:1 to 23:1, 22:1 to 32:1.

The crystallinity of the itraconazole, as well as the size of the itraconazole sub-particles, appears to be important for effective therapy and for reduced toxicity in the lung. Without wishing to be bound by any particular theory, it is believed that smaller sub-particles of itraconazole in crystalline form will dissolve in the airway lining fluid more rapidly than larger particles of itraconazole in the same crystalline form—in part due to the larger amount of surface area. It is also believed that crystalline itraconazole will dissolve more slowly in the airway lining fluid than amorphous itraconazole. Accordingly, the dry powders described herein can be formulated using itraconazole in crystalline particulate form that provide for a desired degree of crystallinity and sub-particle size and can be tailored to achieve desired pharmacokinetic properties while avoiding unacceptable toxicity in the lungs.

The respirable dry particles contain about 1% to about 95% itraconazole by weight (wt %). It is preferred that the respirable dry particle contains an amount of itraconazole so that a therapeutically effective dose can be administered and maintained without the need to inhale large volumes of dry powder more than three times a day. For example, it is preferred that the respirable dry particles contain about 10% to 75%, about 15% to 75%, about 25% to 75%, about 30% to 70%, about 40% to 60%, about 20%, about 50%, or about 70% itraconazole by weight (wt %). The respirable dry particles may contain about 75%, about 80%, about 85%, about 90%, or about 95% itraconazole by weight (wt %). In particular embodiments, the range of itraconazole in the respirable dry particles is about 40% to about 90%, about 55% to about 85%, about 55% to about 75%, or about 65% to about 85%, by weight (wt %). The amount of itraconazole present in the respirable dry particles by weight is also referred to as the “drug load.”

The itraconazole is present in the respirable dry particles in crystalline particulate form (e.g., nano-crystalline). More specifically, in the form of a sub-particle that is about 50 nm to about 5,000 nm (Dv50), preferably, with the itraconazole being at least 50% crystalline. For example, for any desired drug load, the sub-particle size can be about 100 nm, about 300 nm, about 1500 nm, about 80 nm to about 300 nm, about 80 nm to about 250 nm, about 80 nm to about 200 nm, about 100 nm to about 150 nm, about 1200 nm to about 1500 nm, about 1500 nm to about 1750 nm, about 1200 nm to about 1400 nm, or about 1200 nm to about 1350 nm (Dv50). In particular embodiments, the sub-particle is between about 50 nm to about 2500 nm, between about 50 nm and 1000 nm, between about 50 nm and 800 nm, between about 50 nm and 600 nm, between about 50 nm and 500 nm, between about 50 nm and 400 nm, between about 50 nm and 300 nm, between about 50 nm and 200 nm, or between about 100 nm and 300 nm. In addition, for any desired drug load and sub-particle size, the degree of itraconazole crystallinity can be at least about 50%, at least about 60%, at least about 70%, at least about 80%, at least about 90%, at least about 95%, or about 100% crystalline. Preferably, the itraconazole is about 100% crystalline.

The itraconazole in crystalline particulate form can be prepared in any desired sub-particle size using a suitable method, including polysorbate 80 if desired, such as by wet milling, jet milling or other suitable method.

The respirable dry particles include polysorbate 80 as a stabilizer. The polysorbate 80 helps maintain the desired size of the itraconazole in crystalline particulate form during wet milling, in spray drying feedstock, and aids in wetting and dispersing and maintaining the physical stability of the itraconazole crystalline particulate suspension. It is preferred to use as little polysorbate 80 as is needed to achieve the aforementioned benefits. The amount of polysorbate 80 is typically in a fixed ratio to the amount of itraconazole present in the dry particle and is greater than 10:1 itraconazole:polysorbate 80 (wt:wt), greater than 10:1 to 25:1, 11:1 to 35:1, 10.5:1 to 14.5:1, 11:1 to 31:1, greater than 12:1, 11:1 to 15:1, 11.5:1 to 14:1, 13:1 to 16:1, or 15:1 to 19.5:1, 19:1 to 25:1, 20.5:1 to 23:1, 22:1 to 32:1. Alternatively, the ratio of itraconazole:polysorbate 80 (wt:wt) in the dry particles can be greater than or equal to 11.5:1, greater than or equal to 12:1, greater than or equal to 14:1, greater than or equal to 15:1, greater than or equal to 16:1, greater than or equal to 17:1, greater than or equal to 18:1, greater than or equal to 19:1; about 11:1, about 12:1, about 13:1, about 14:1, about 15:1, about 18:1, about 19.5:1, or about 22:1. In some embodiments, the amount of polysorbate 80 that is present in the dry particles can be in a range of about 0.1% to less than 10% by weight (wt %) or in a range of about 1% to about 9% by weight. In particular embodiments, the range is about 1% to about 15%, about 4% to about 10%, or about 5% to about 8% by weight (wt %). It is generally preferred that the respirable dry particles contain less than about 10% polysorbate 80 by weight (wt %), such as 7 wt %, 5 wt % or 1 wt %. Alternatively, the respirable dry particles contain about 5 wt %, about 6 wt %, about 7 wt %, about 7.5 wt %, about 8 wt %, or about 10% polysorbate 80. It is particularly preferred that respirable dry particles contain less than about 8 wt % polysorbate 80. In contrast to the prior art, which uses polysorbate 80 to prevent the onset of crystallization in the produced dry powder, the polysorbate 80 in the present invention is added to stabilize a colloidal suspension of the crystalline itraconazole in an anti-solvent.

The respirable dry particles also include any suitable and desired amount of one or more excipients. The dry particles can contain a total excipient content of about 10 wt % to about 99 wt %, with about 25 wt % to about 85 wt %, or about 40 wt % to about 55 wt % being more typical. The dry particles can contain a total excipient content of about 1 wt %, about 2 wt %, about 4 wt %, about 6 wt %, about 8 wt %, or less than about 10 wt %. In particular embodiments, the range is about 5% to about 50%, about 15% to about 50%, about 25% to about 50%, about 5% to about 40%, about 5% to about 30%, about 5% to about 20%, or about 5% to about 15%. In other embodiments, the range of excipient is about 1% to about 9%, about 2% to about 9%, about 3% to about 9%, about 4% to about 9%, about 5% to about 9%, about 1% to about 8%, about 2% to about 8%, about 3% to about 8%, about 4% to about 8%, about 5% to about 8%, about 1% to about 7%, about 2% to about 7%, about 3% to about 7%, about 4% to about 7%, about 5% to about 7%, about 1% to about 6%, about 2% to about 6%, about 3% to about 6%, or about 1% to about 5%.

Many excipients are well-known in the art and can be included in the dry powders and dry particles described herein. Pharmaceutically acceptable excipients that are particularly preferred for the dry powders and dry particles described herein include monovalent and divalent metal cation salts, carbohydrates, sugar alcohols and amino acids.

Suitable monovalent metal cation salts, include, for example, sodium salts and potassium salts. Suitable sodium salts that can be present in the respirable dry particles of the invention include, for example, sodium chloride, sodium citrate, sodium sulfate, sodium lactate, sodium acetate, sodium bicarbonate, sodium carbonate, sodium stearate, sodium ascorbate, sodium benzoate, sodium biphosphate, sodium phosphate, sodium bisulfite, sodium borate, sodium gluconate, sodium metasilicate and the like.

Suitable potassium salts include, for example, potassium chloride, potassium bromide, potassium iodide, potassium bicarbonate, potassium nitrite, potassium persulfate, potassium sulfite, potassium bisulfite, potassium phosphate, potassium acetate, potassium citrate, potassium glutamate, dipotassium guanylate, potassium gluconate, potassium malate, potassium ascorbate, potassium sorbate, potassium succinate, potassium sodium tartrate and any combination thereof.

Suitable divalent metal cation salts, include magnesium salts and calcium salts. Suitable magnesium salts include, for example, magnesium lactate, magnesium fluoride, magnesium chloride, magnesium bromide, magnesium iodide, magnesium phosphate, magnesium sulfate, magnesium sulfite, magnesium carbonate, magnesium oxide, magnesium nitrate, magnesium borate, magnesium acetate, magnesium citrate, magnesium gluconate, magnesium maleate, magnesium succinate, magnesium malate, magnesium taurate, magnesium orotate, magnesium glycinate, magnesium naphthenate, magnesium acetylacetonate, magnesium formate, magnesium hydroxide, magnesium stearate, magnesium hexafluorsilicate, magnesium salicylate or any combination thereof.

Suitable calcium salts include, for example, calcium chloride, calcium sulfate, calcium lactate, calcium citrate, calcium carbonate, calcium acetate, calcium phosphate, calcium alginate, calcium stearate, calcium sorbate, calcium gluconate and the like.

A preferred sodium salt is sodium sulfate. A preferred sodium salt is sodium chloride. A preferred sodium salt is sodium citrate. A preferred magnesium salt is magnesium lactate.

Carbohydrate excipients that are useful in this regard include the mono- and polysaccharides, sugar alcohols, dextrans, dextrins, and cyclodextrins, amongst others. Representative monosaccharides include dextrose (anhydrous and the monohydrate; also referred to as glucose and glucose monohydrate), galactose, D-mannose, sorbose and the like. Representative disaccharides include lactose, maltose, sucrose, trehalose and the like. Representative trisaccharides include raffinose and the like. Other carbohydrate excipients including dextran, maltodextrin and cyclodextrins, such as 2-hydroxypropyl-beta-cyclodextrin can be used as desired. A preferred carbohydrate is maltodextrin. Representative sugar alcohols include mannitol, sorbitol and the like. A preferred sugar alcohol is mannitol. Preferred carbohydrates are mannitol, lactose, maltodextrin and trehalose.

Suitable amino acid excipients include any of the naturally occurring amino acids that form a powder under standard pharmaceutical processing techniques and include the non-polar (hydrophobic) amino acids and polar (uncharged, positively charged and negatively charged) amino acids, such amino acids are of pharmaceutical grade and are generally regarded as safe (GRAS) by the U.S. Food and Drug Administration. Representative examples of non-polar amino acids include alanine, isoleucine, leucine, methionine, phenylalanine, proline, tryptophan and valine. Representative examples of polar, uncharged amino acids include cysteine, glycine, glutamine, serine, threonine, and tyrosine. Representative examples of polar, positively charged amino acids include arginine, histidine and lysine. Representative examples of negatively charged amino acids include aspartic acid and glutamic acid. A preferred amino acid is leucine.

In one aspect, the respirable dry particles comprise leucine as one of the one or more excipients in an amount of about 1% to about 9%, about 2% to about 9%, about 3% to about 9%, about 4% to about 9%, about 5% to about 9%, about 1% to about 8%, about 2% to about 8%, about 3% to about 8%, about 4% to about 8%, about 5% to about 8%, about 1% to about 7%, about 2% to about 7%, about 3% to about 7%, about 4% to about 7%, about 5% to about 7%, about 1% to about 6%, about 2% to about 6%, about 3% to about 6%, about 1% to about 5%, about 1%, about 2%, about 3%, about 4%, about 5%, about 6%, about 7%, about 9%, or about 10%.

The dry particles described herein contain 1) itraconazole in crystalline particulate form, 2) polysorbate 80, and optionally 3) one or more excipients. In some aspects, the dry particles contain a first excipient that is a monovalent or divalent metal cation salt, and a second excipient that is an amino acid, carbohydrate or sugar alcohol. For example, the first excipient can be a sodium salt or a magnesium salt, and the second excipient can be an amino acid (such as leucine). In more particular examples, the first excipient can be sodium sulfate, sodium chloride or magnesium lactate, and the second excipient can be leucine. Even more particularly, the first excipient can be sodium sulfate and the second excipient can be leucine. In another example, the first excipient can be a sodium salt or a magnesium salt, and the second excipient can be a sugar alcohol (such as mannitol). In more particular examples, the first excipient can be sodium sulfate, sodium chloride or magnesium lactate, and the second excipient can be mannitol. In other examples, the dry particles include itraconazole in crystalline particulate form, polysorbate 80 and one excipient, for example a sodium salt, a magnesium salt or an amino acid (e.g. leucine). In this aspect, the dry powder formulation does not comprise lactose.

In one aspect, the invention relates to dry powder formulations comprising respirable dry particles comprising 1) itraconazole in crystalline particulate form, 2) polysorbate 80, and 3) one or more excipients, wherein the ratio of itraconazole to polysorbate 80 in the nanoparticle suspension used in the feedstock is greater than 10:1, greater than 10:1 to 25:1, 11:1 to 35:1, 10.5:1 to 14.5:1, 11:1 to 31:1, greater than 12:1, 11:1 to 15:1, 11.5:1 to 14:1, 13:1 to 16:1, 15:1 to 19.5:1, 19:1 to 25:1, 20.5:1 to 23:1, or 22:1 to 32:1, with the proviso that the dry powder formulation does not comprise: 20% Itraconazole, 39% sodium sulfate, 39% mannitol, and 2% polysorbate 80; 50% Itraconazole, 22.5% sodium sulfate, 22.5% mannitol, and 5% polysorbate 80; 20% Itraconazole, 62.4% sodium chloride, 15.6% leucine, and 2% polysorbate 80; 50% Itraconazole, 36% sodium sulfate, 9% leucine, and 5% polysorbate 80; 20% Itraconazole, 66.3% magnesium lactate, 11.7% leucine, and 2% polysorbate 80; 50% Itraconazole, 38.25% magnesium lactate, 6.75% leucine, and 5% polysorbate 80; 50% Itraconazole, 35% sodium sulfate, 10% leucine, and 5% polysorbate 80; 50% Itraconazole, 35% sodium sulfate, 10% leucine, and less than 5% polysorbate 80; 50% Itraconazole, 35% sodium sulfate, 13.75% leucine, and 1.25% polysorbate 80; 50% Itraconazole, 37% sodium sulfate, 8% leucine, and 5% polysorbate 80; 60% Itraconazole, 26% sodium sulfate, 8% leucine, and 6% polysorbate 80; 70% Itraconazole, 15% sodium, 8% leucine, and 7% polysorbate 80; 75% Itraconazole, 9.5% sodium sulfate, 8% leucine, and 7.5% polysorbate 80; 80% Itraconazole, 4% sodium sulfate, 8% leucine, and 8% polysorbate 80; 80% Itraconazole, 10% sodium sulfate, 2% leucine, and 8% polysorbate 80; 80% Itraconazole, 11% sodium sulfate, 1% leucine, and 8% polysorbate 80; or 80% Itraconazole, 11% sodium sulfate, 1% leucine, and 8% polysorbate 80.

The dry powders and/or respirable dry particles are preferably small, mass dense, and dispersible. To measure volumetric median geometric diameter (VMGD), a laser diffraction system may be used, e.g., a Spraytec system (particle size analysis instrument, Malvern Instruments) and a HELOS/RODOS system (laser diffraction sensor with dry dispensing unit, Sympatec GmbH). The respirable dry particles have a VMGD as measured by laser diffraction at the dispersion pressure setting (also called regulator pressure) of 1.0 bar at a maximum orifice ring pressure using a HELOS/RODOS system of about 10 microns or less, about 5 microns or less, about 4 μm or less, about 3 μm or less, about 1 μm to about 5 μm, about 1 82 m to about 4 μm, about 1.5 μm to about 3.5 μm, about 2 μm to about 5 μm, about 2 μm to about 4 μm, or about 2 μm to about 3 μm. Preferably, the VMGD is about 5 microns or less or about 4 μm or less. In one aspect, the dry powders and/or respirable dry particles have a minimum VMGD of about 0.5 microns or about 1.0 micron.

The dry powders and/or respirable dry particles preferably have 1 bar/4 bar dispersibility ratio and/or 0.5 bar/4 bar dispersibility ratio of less than about 2.0 (e.g., about 0.9 to less than about 2), about 1.7 or less (e.g., about 0.9 to about 1.7) about 1.5 or less (e.g., about 0.9 to about 1.5), about 1.4 or less (e.g., about 0.9 to about 1.4), or about 1.3 or less (e.g., about 0.9 to about 1.3), and preferably have a 1 bar/4 bar and/or a 0.5 bar/4 bar of about 1.5 or less (e.g., about 1.0 to about 1.5), and/or about 1.4 or less (e.g., about 1.0 to about 1.4).

The dry powders and/or respirable dry particles preferably have a tap density of at least about 0.2 g/cm³, of at least about 0.25 g/cm³, a tap density of at least about 0.3 g/cm³, of at least about 0.35 g/cm³, a tap density of at least 0.4 g/cm³. For example, the dry powders and/or respirable dry particles have a tap density of greater than 0.4 g/cm³ (e.g., greater than 0.4 g/cm³ to about 1.2 g/cm³), a tap density of at least about 0.45 g/cm³ (e.g., about 0.45 g/cm³ to about 1.2 g/cm³), at least about 0.5 g/cm³ (e.g., about 0.5 g/cm³ to about 1.2 g/cm³), at least about 0.55 g/cm³ (e.g., about 0.55 g/cm³ to about 1.2 g/cm³), at least about 0.6 g/cm³ (e.g., about 0.6 g/cm³ to about 1.2 g/cm³) or at least about 0.6 g/cm³ to about 1.0 g/cm³. Alternatively, the dry powders and/or respirable dry particles preferably have a tap density of about 0.01 g/cm³ to about 0.5 g/cm³, about 0.05 g/cm³ to about 0.5 g/cm³, about 0.1 g/cm³ to about 0.5 g/cm³, about 0.1 g/cm³ to about 0.4 g/cm³, or about 0.1 g/cm³ to about 0.4 g/cm³. Alternatively, the dry powders and/or respirable dry particles have a tap density of about 0.15 g/cm³ to about 1.0 g/cm³. Alternatively, the dry powders and/or respirable dry particles have a tap density of about 0.3 g/cm³ to about 0.8 g/cm³.

The dry powders and/or respirable dry particles have a bulk density of at least about 0.1 g/cm³, or at least about 0.8 g/cm³. For example, the dry powders and/or respirable dry particles have a bulk density of about 0.1 g/cm³ to about 0.6 g/cm³, about 0.2 g/cm³ to about 0.7 g/cm³, about 0.3 g/cm³ to about 0.8 g/cm³.

The respirable dry particles, and the dry powders when the dry powders are respirable dry powders, preferably have an MMAD of less than 10 microns, preferably an MMAD of about 5 microns or less, or about 4 microns or less. In one aspect, the respirable dry powders and/or respirable dry particles preferably have a minimum MMAD of about 0.5 microns, or about 1.0 micron. In one aspect, the respirable dry powders and/or respirable dry particles preferably have a minimum MMAD of about 2.0 microns, about 3.0 microns, or about 4.0 microns.

The dry powders and/or respirable dry particles preferably have a FPF of less than about 5.6 microns (FPF<5.6 μm) of the total dose of at least about 35%, preferably at least about 45%, at least about 60%, between about 45% to about 80%, or between about 60% and about 80%.

The dry powders and/or respirable dry particles preferably have a FPF of less than about 3.4 microns (FPF<3.4 μm) of the total dose of at least about 20%, preferably at least about 25%, at least about 30%, at least about 40%, between about 25% and about 60%, or between about 40% and about 60%.

The dry powders and/or respirable dry particles preferably have a total water and/or solvent content of up to about 15% by weight, up to about 10% by weight, up to about 5% by weight, up to about 1%, or between about 0.01% and about 1%, or may be substantially free of water or other solvent.

The dry powders and/or respirable dry particles preferably may be administered with low inhalation energy. In order to relate the dispersion of powder at different inhalation flow rates, volumes, and from inhalers of different resistances, the energy required to perform the inhalation maneuver may be calculated. Inhalation energy can be calculated from the equation E=R²Q²V where E is the inhalation energy in Joules, R is the inhaler resistance in kPa^{1/2}/LPM, Q is the steady flow rate in L/min and V is the inhaled air volume in L.

Healthy adult populations are predicted to be able to achieve inhalation energies ranging from 2.9 Joules for comfortable inhalations to 22 Joules for maximum inhalations by using values of peak inspiratory flow rate (PIFR) measured by Clarke et al. (Journal of Aerosol Med, 6(2), p.99-110, 1993) for the flow rate Q from two inhaler resistances of 0.02 and 0.055 kPa^{1/2}/LPM, with an inhalation volume of 2 L based on both FDA guidance documents for dry powder inhalers and on the work of Tiddens et al. (Journal of Aerosol Med, 19(4), p.456-465, 2006) who found adults averaging 2.2 L inhaled volume through a variety of DPIs.

Mild, moderate and severe adult COPD patients are predicted to be able to achieve maximum inhalation energies of 5.1 to 21 Joules, 5.2 to 19 Joules, and 2.3 to 18 Joules respectively. This is again based on using measured PIFR values for the flow rate Q in the equation for inhalation energy. The PIFR achievable for each group is a function of the inhaler resistance that is being inhaled through. The work of Broeders et al. (Eur Respir J, 18, p.780-783, 2001) was used to predict maximum and minimum achievable PIFR through two dry powder inhalers of resistances 0.021 and 0.032 kPa^{1/2}/LPM for each.

Similarly, adult asthmatic patients are predicted to be able to achieve maximum inhalation energies of 7.4 to 21 Joules based on the same assumptions as the COPD population and PIFR data from Broeders et al.

Healthy adults and children, COPD patients, asthmatic patients ages 5 and above, and CF patients, for example, are capable of providing sufficient inhalation energy to empty and disperse the dry powder formulations of the invention.

The dry powders and/or respirable dry particles are preferably characterized by a high emitted dose, such as a CEPM of at least 75%, at least 80%, at least 85%, at least 90%, at least 95%, from a passive dry powder inhaler subject to a total inhalation energy of about 5 Joules, about 3.5 Joules, about 2.4 Joules, about 2 Joules, about 1 Joule, about 0.8 Joules, about 0.5 Joules, or about 0.3 Joules is applied to the dry powder inhaler. The receptacle holding the dry powders and/or respirable dry particles may contain about 5 mg, about 7.5 mg, about 10 mg, about 15 mg, about 20 mg, or about 30 mg. In one aspect, the dry powders and/or respirable dry particles are characterized by a CEPM of 80% or greater and a VMGD of 5 microns or less when emitted from a passive dry powder inhaler having a resistance of about 0.036 sqrt(kPa)/liters per minute under the following conditions: an air flow rate of 30 LPM, run for 3 seconds using a size 3 capsule that contains a total mass of 10 mg. In another aspect, the dry powders and/or respirable dry particles are characterized by a CEPM of 80% or greater and a VMGD of 5 microns or less when emitted from a passive dry powder inhaler having a resistance of about 0.036 sqrt(kPa)/liters per minute under the following conditions: an air flow rate of 20 LPM, run for 3 seconds using a size 3 capsule that contains a total mass of 10 mg. In a further aspect, the dry powders and/or respirable dry particles are characterized by a CEPM of 80% or greater and a VMGD of 5 microns or less when emitted from a passive dry powder inhaler having a resistance of about 0.036 sqrt(kPa)/liters per minute under the following conditions: an air flow rate of 15 LPM, run for 4 seconds using a size 3 capsule that contains a total mass of 10 mg.

The dry powder can fill the unit dose container, or the unit dose container can be at least 2% full, at least 5% full, at least 10% full, at least 20% full, at least 30% full, at least 40% full, at least 50% full, at least 60% full, at least 70% full, at least 80% full, or at least 90% full. The unit dose container can be a capsule (e.g., size 000, 00, 0E, 0, 1, 2, 3, and 4, with respective volumetric capacities of 1.37 ml, 950 μl, 770 μl, 680 μl, 480 μl, 360 μl, 270 μl, and 200 μl). The capsule can be at least about 2% full, at least about 5% full, at least about 10% full, at least about 20% full, at least about 30% full, at least about 40% full, or at least about 50% full. The unit dose container can be a blister. The blister can be packaged as a single blister or as part of a set of blisters, for example, 7 blisters, 14 blisters, 28 blisters or 30 blisters. The one or more blister can be preferably at least 30% full, at least 50% full or at least 70% full.

An advantage of the invention is the production of powders that disperse well across a wide range of flow rates and are relatively flowrate independent. The dry powders and/or respirable dry particles of the invention enable the use of a simple, passive DPI for a wide patient population.

In particular aspects, the invention relates to dry powders and/or respirable dry particles that comprise itraconazole in crystalline particulate form (e.g., particles of about 80 nm to about 1750 rim volume median diameter (Dv50), such as about 60 rim to about 175 nm Dv50, about 150 nm to about 400 nm Dv50 or about 1200 nm to about 1750 nm Dv50; alternatively, 50 nm to 800 nm Dv50), a stabilizer, and optionally one or more excipients. Particular dry powders and respirable dry particles have the following formulations shown in Table 1. The dry powders and/or respirable dry particles described herein are preferably characterized by: 1) a VMGD at 1 bar as measured using a HELOS/RODOS system of about 10 microns or less, preferably about 5 microns or less; 2) a 1 bar/4 bar dispersibility ratio and/or a 0.5 bar/4 bar dispersibility ratio of about 1.5 or less, about 1.4 or less or about 1.3 or less; 3) a MMAD of about 10 microns or less, preferably about 5 microns or less; 4) a FPF<5.6 μm of the total dose of at least about 45% or at least about 60%; and/or 5) a FPF<3.4 μm of the total dose of at least about 25% or at least about 40%. If desired, the dry powders and/or respirable dry particles are further characterized by a tap density of about 0.2 g/cm³ or greater, about 0.3 g/cm³ or greater, about 0.4 g/cm³ or greater, greater than 0.4 g/cm³, about 0.45 g/cm³ or greater or about 0.5 g/cm³ or greater.

TABLE 1

| | | | | | Itraconazole |
|---|---|---|---|---|---|
| | | | Polysorbate | | subparticle |
| | Itraconazole | Excipients | 80 (PS 80) | Itraconazole:PS | size range |
| Formulation | (wt %) | (wt %) | (wt %) | 80 ratio | (Dv50 nm) |
| I | Itraconazole | Sodium | PS 80 1.66% | 12:1 | 60-175 |
| | 20.0% | Sulfate | | | |
| | | 39.2%, | | | |
| | | Mannitol | | | |
| | | 39.2% | | | |
| II | Itraconazole | Sodium | PS 80 4.17% | 12:1 | 60-175 |
| | 50.0% | Sulfate | | | |
| | | 22.9%, | | | |
| | | Mannitol | | | |
| | | 22.9% | | | |
| III | Itraconazole | Sodium | PS 80 4.17% | 12:1 | 60-175 |
| | 50.0% | Sulfate | | | |
| | | 45.8% | | | |
| IV | Itraconazole | Sodium | PS 80 6.67% | 12:1 | 60-175 |
| | 80.0% | Sulfate | | | |
| | | 6.66%, | | | |
| | | Mannitol | | | |
| | | 6.67% | | | |
| V | Itraconazole | Sodium | PS 80 6.67% | 12:1 | 60-175 |
| | 80.0% | Sulfate | | | |
| | | 13.3% | | | |
| VI | Itraconazole | N/A | PS 80 7.69% | 12:1 | 60-175 |
| | 92.3% | | | | |
| VII | Itraconazole | Sodium | PS 80 1.00% | 20:1 | 60-175 |
| | 20.0% | Sulfate | | | |
| | | 39.5%, | | | |
| | | Mannitol | | | |
| | | 39.5% | | | |
| VIII | Itraconazole | Sodium | PS 80 2.50% | 20:1 | 60-175 |
| | 50.0% | Sulfate | | | |
| | | 23.8%, | | | |
| | | Mannitol | | | |
| | | 23.8% | | | |
| IX | Itraconazole | Sodium | PS 80 4.00% | 20:1 | 60-175 |
| | 80.0% | Sulfate | | | |
| | | 8.00%, | | | |
| | | Mannitol | | | |
| | | 8.00% | | | |
| X | Itraconazole | Sodium | PS 80 1.66% | 12:1 | 60-175 |
| | 20.0% | Sulfate | | | |
| | | 60.9%, | | | |
| | | Leucine | | | |
| | | 17.4% | | | |
| XI | Itraconazole | Sodium | PS 80 4.16% | 12:1 | 60-175 |
| | 50.0% | Sulfate | | | |
| | | 35.7%, | | | |
| | | Leucine | | | |
| | | 10.2% | | | |
| XII | Itraconazole | Sodium | PS 80 5.00% | 12:1 | 60-175 |
| | 60.0% | Sulfate | | | |
| | | 27.2%, | | | |
| | | Leucine | | | |
| | | 7.78% | | | |
| XIII | Itraconazole | Sodium | PS 80 5.83% | 12:1 | 60-175 |
| | 70.0% | Sulfate | | | |
| | | 18.8%, | | | |
| | | Leucine | | | |
| | | 5.37% | | | |
| XIV | Itraconazole | Sodium | PS 80 6.67% | 12:1 | 60-175 |
| | 80.0% | Sulfate | | | |
| | | 10.4%, | | | |
| | | Leucine | | | |
| | | 2.96% | | | |
| XV | Itraconazole | Sodium | PS 80 6.67% | 12:1 | 60-175 |
| | 80.0% | Sulfate | | | |
| | | 6.67%, | | | |
| | | Leucine | | | |
| | | 6.66% | | | |
| XVI | Itraconazole | Sodium | PS 80 6.67% | 12:1 | 60-175 |
| | 80.0% | Sulfate | | | |
| | | 2.96%, | | | |
| | | Leucine | | | |
| | | 10.4% | | | |
| XVII | Itraconazole | Sodium | PS 80 1.00% | 20:1 | 60-175 |
| | 20.0% | Sulfate | | | |
| | | 61.4%, | | | |
| | | Leucine | | | |
| | | 17.6% | | | |
| XVIII | Itraconazole | Sodium | PS 80 2.50% | 20:1 | 60-175 |
| | 50.0% | Sulfate | | | |
| | | 36.9%, | | | |
| | | Leucine | | | |
| | | 10.6% | | | |
| XIX | Itraconazole | Sodium | PS 80 2.50% | 20:1 | 60-175 |
| | 50.0% | Sulfate | | | |
| | | 47.5% | | | |
| XX | Itraconazole | Sodium | PS 80 3.00% | 20:1 | 60-175 |
| | 60.0% | Sulfate | | | |
| | | 28.8%, | | | |
| | | Leucine | | | |
| | | 8.20% | | | |
| XXI | Itraconazole | Sodium | PS 80 3.50% | 20:1 | 60-175 |
| | 70.0% | Sulfate | | | |
| | | 20.6%, | | | |
| | | Leucine | | | |
| | | 5.89% | | | |
| XXI | Itraconazole | Sodium | PS 80 4.00% | 20:1 | 60-175 |
| | 80.0% | Sulfate | | | |
| | | 12.4%, | | | |
| | | Leucine | | | |
| | | 3.56% | | | |
| XXII | Itraconazole | N/A | PS 80 4.76% | 20:1 | 60-175 |
| | 95.2% | | | | |
| XXIII | Itraconazole | Sodium | PS 80 0.667% | 30:1 | 60-175 |
| | 20.0% | Sulfate | | | |
| | | 61.7%, | | | |
| | | Leucine | | | |
| | | 17.6% | | | |
| XXIV | Itraconazole | Sodium | PS 80 1.67% | 30:1 | 60-175 |
| | 50.0% | Sulfate | | | |
| | | 37.6%, | | | |
| | | Leucine | | | |
| | | 10.7% | | | |
| XXV | Itraconazole | Sodium | PS 80 2.00% | 30:1 | 60-175 |
| | 60% | Sulfate | | | |
| | | 29.6%, | | | |
| | | Leucine | | | |
| | | 8.44% | | | |
| XXVI | Itraconazole | Sodium | PS 80 2.33% | 30:1 | 60-175 |
| | 70% | Sulfate | | | |
| | | 21.5%, | | | |
| | | Leucine | | | |
| | | 6.15% | | | |
| XXVII | Itraconazole | Sodium | PS 80 2.67% | 30:1 | 60-175 |
| | 80% | Sulfate | | | |
| | | 13.5%, | | | |
| | | Leucine | | | |
| | | 3.85% | | | |

In another aspect, the invention relates to a dry powder formulation comprising about 50% to about 80% Itraconazole, 9% or less leucine, about 20% to about 40% sodium sulfate, and polysorbate 80 in a ratio of greater than 10:1 Itraconazole:polysorbate 80. The dry powders and/or respirable dry particles are preferably small, mass dense, and dispersible. To measure volumetric median geometric diameter (VMGD), a laser diffraction system may be used, e.g., a Spraytec system (particle size analysis instrument, Malvern Instruments) and a HELOS/RODOS system (laser diffraction sensor with dry dispensing unit, Sympatec GmbH). The respirable dry particles have a VMGD as measured by laser diffraction at the dispersion pressure setting (also called regulator pressure) of 1.0 bar at a maximum orifice ring pressure using a HELOS/RODOS system of about 10 microns or less, about 5 microns or less, about 4 μm or less, about 3 μm or less, about 1 μm to about 5 μm, about 1 μm to about 4 μm, about 1.5 μm to about 3.5 μm, about 2 μm to about 5 μm, about 2 μm to about 4 μm, or about 2 μm to about 3 μm. Preferably, the VMGD is about 5 microns or less or about 4 μm or less. In one aspect, the dry powders and/or respirable dry particles have a minimum VMGD of about 0.5 microns or about 1.0 micron.

The dry powders and/or respirable dry particles described by any of the ranges or specifically disclosed formulations, characterized in the previous paragraph, may be filled into a receptacle, for example a capsule or a blister. When the receptacle is a capsule, the capsule is, for example, a size 2 or a size 3 capsule, and is preferably a size 3 capsule. The capsule material may be, for example, gelatin or HPMC (Hydroxypropyl methylcellulose), and is preferably HPMC.

The dry powder and/or respirable dry particles described and characterized above may be contained in a dry powder inhaler (DPI). The DPI may be a capsule-based DPI or a blister-based DPI, and is preferably a capsule-based DPI. More preferably, the dry powder inhaler is selected from the RS01 family of dry powder inhalers (Plastiape S.p.A., Italy). More preferably, the dry powder inhaler is selected from the RS01 HR or the RS01 UHR2. Most preferably, the dry powder inhaler is the RS01 HR.

### Methods for Preparing Dry Powders and Dry Particles

The respirable dry particles and dry powders can be prepared using any suitable method, with the proviso that the dry powder formulation cannot be an extemporaneous dispersion. Many suitable methods for preparing dry powders and/or respirable dry particles are conventional in the art, and include single and double emulsion solvent evaporation, spray drying, spray-freeze drying, milling (e.g., jet milling), blending, solvent extraction, solvent evaporation, phase separation, simple and complex coacervation, interfacial polymerization, suitable methods that involve the use of supercritical carbon dioxide (CO₂), sonocrystalliztion, nanoparticle aggregate formation and other suitable methods, including combinations thereof. Respirable dry particles can be made using methods for making microspheres or microcapsules known in the art. These methods can be employed under conditions that result in the formation of respirable dry particles with desired aerodynamic properties (e.g., aerodynamic diameter and geometric diameter). If desired, respirable dry particles with desired properties, such as size and density, can be selected using suitable methods, such as sieving.

Suitable methods for selecting respirable dry particles with desired properties, such as size and density, include wet sieving, dry sieving, and aerodynamic classifiers (such as cyclones).

The respirable dry particles are preferably spray dried. Suitable spray-drying techniques are described, for example, by K. Masters in “Spray Drying Handbook”, John Wiley & Sons, New York (1984). Generally, during spray-drying, heat from a hot gas such as heated air or nitrogen is used to evaporate a solvent from droplets formed by atomizing a continuous liquid feed. When hot air is used, the moisture in the air is at least partially removed before its use. When nitrogen is used, the nitrogen gas can be run “dry”, meaning that no additional water vapor is combined with the gas. If desired the moisture level of the nitrogen or air can be set before the beginning of spray dry run at a fixed value above “dry” nitrogen. If desired, the spray drying or other instruments, e.g., jet milling instrument, used to prepare the dry particles can include an inline geometric particle sizer that determines a geometric diameter of the respirable dry particles as they are being produced, and/or an inline aerodynamic particle sizer that determines the aerodynamic diameter of the respirable dry particles as they are being produced.

For spray drying, solutions, emulsions or suspensions that contain the components of the dry particles to be produced in a suitable solvent (e.g., aqueous solvent, organic solvent, aqueous-organic mixture or emulsion) are distributed to a drying vessel via an atomization device. For example, a nozzle or a rotary atomizer may be used to distribute the solution or suspension to the drying vessel. The nozzle can be a two-fluid nozzle, which can be in an internal mixing setup or an external mixing setup. Alternatively, a rotary atomizer having a 4- or 24-vaned wheel may be used. Examples of suitable spray dryers that can be outfitted with a rotary atomizer and/or a nozzle, include, a Mobile Minor Spray Dryer or the Model PSD-1, both manufactured by GEA Niro, Inc. (Denmark), Büchi B-290 Mini Spray Dryer (BÜCHI Labortechnik AG, Flawil, Switzerland), ProCepT Formatrix R&D spray dryer (ProCepT nv, Zelzate, Belgium), among several other spray dryer options. Actual spray drying conditions will vary depending, in part, on the composition of the spray drying solution or suspension and material flow rates. The person of ordinary skill will be able to determine appropriate conditions based on the compositions of the solution, emulsion or suspension to be spray dried, the desired particle properties and other factors. In general, the inlet temperature to the spray dryer is about 90° C. to about 300° C. The spray dryer outlet temperature will vary depending upon such factors as the feed temperature and the properties of the materials being dried. Generally, the outlet temperature is about 50° C. to about 150° C. If desired, the respirable dry particles that are produced can be fractionated by volumetric size, for example, using a sieve, or fractioned by aerodynamic size, for example, using a cyclone, and/or further separated according to density using techniques known to those of skill in the art.

To prepare the respirable dry particles of the invention, generally, an emulsion or suspension that contains the desired components of the dry powder (i.e., a feedstock) is prepared and spray dried under suitable conditions. Preferably, the dissolved or suspended solids concentration in the feedstock is at least about 1 g/L, at least about 2 g/L, at least about 5 g/L, at least about 10 g/L, at least about 15 g/L, at least about 20 g/L, at least about 30 g/L, at least about 40 g/L, at least about 50 g/L, at least about 60 g/L, at least about 70 g/L, at least about 80 g/L, at least about 90 g/L or at least about 100 g/L. The feedstock can be provided by preparing a single solution, suspension or emulsion by dissolving, suspending, or emulsifying suitable components (e.g., salts, excipients, other active ingredients) in a suitable solvent. The solution, emulsion or suspension can be prepared using any suitable methods, such as bulk mixing of dry and/or liquid components or static mixing of liquid components to form a combination. For example, a hydrophilic component (e.g., an aqueous solution) and a hydrophobic component (e.g., an organic solution) can be combined using a static mixer to form a combination. The combination can then be atomized to produce droplets, which are dried to form respirable dry particles. Preferably, the atomizing step is performed immediately after the components are combined in the static mixer. Alternatively, the atomizing step is performed on a bulk mixed solution.

The feedstock can be prepared using any solvent in which the itraconazole in particulate form has low solubility, such as an organic solvent, an aqueous solvent or mixtures thereof. Suitable organic solvents that can be employed include but are not limited to alcohols such as, for example, ethanol, methanol, propanol, isopropanol, butanols, and others. Other organic solvents include but are not limited to tetrahydrofuran (THF), perfluorocarbons, dichloromethane, chloroform, ether, ethyl acetate, methyl tert-butyl ether and others. Co-solvents that can be employed include an aqueous solvent and an organic solvent, such as, but not limited to, the organic solvents as described above. Aqueous solvents include water and buffered solutions. A preferred solvent is water.

Various methods (e.g., static mixing, bulk mixing) can be used for mixing the solutes and solvents to prepare feedstocks, which are known in the art. If desired, other suitable methods of mixing may be used. For example, additional components that cause or facilitate the mixing can be included in the feedstock. For example, carbon dioxide produces fizzing or effervescence and thus can serve to promote physical mixing of the solute and solvents.

The feedstock or components of the feedstock can have any desired pH, viscosity or other properties. If desired, a pH buffer can be added to the solvent or co-solvent or to the formed mixture. Generally, the pH of the mixture ranges from about 3 to about 8.

Dry powder and/or respirable dry particles can be fabricated and then separated, for example, by filtration or centrifugation by means of a cyclone, to provide a particle sample with a preselected size distribution. For example, greater than about 30%, greater than about 40%, greater than about 50%, greater than about 60%, greater than about 70%, greater than about 80%, or greater than about 90% of the respirable dry particles in a sample can have a diameter within a selected range. The selected range within which a certain percentage of the respirable dry particles fall can be, for example, any of the size ranges described herein, such as between about 0.1 to about 3 microns VMGD.

The suspension may be a nano-suspension, similar to an intermediate for making dry powder containing nano-crystalline drug.

The dry powder may be a drug embedded in a matrix material, such as sodium sulfate and leucine. Optionally, the dry powder may be spray dried such that the dry particles are small, dense, and dispersible.

The dry powders can consist solely of the respirable dry particles described herein without other carrier or excipient particles (referred to as “neat powders”).

In a preferred embodiment, the dry powders do not contain carrier particles. In one aspect, the crystalline itraconazole particles are embedded in a matrix comprising excipient and/or stabilizer. The dry powder may comprise respirable dry particles of uniform content, wherein each particle contains crystalline itraconazole. Thus, as used herein, “uniform content” means that every respirable particle contains some amount of itraconazole in crystalline particulate form, polysorbate 80, and excipient.

The dry powders can comprise respirable dry particles wherein at least 98%, at least 99%, or substantially all of the particles (by weight) contain itraconazole.

The dry powders can comprise crystalline itraconazole particles distributed throughout a matrix comprising one or more excipients. The excipients can comprise any number of salts, sugars, lipids, amino acids, surfactants, polymers, or other components suitable for pharmaceutical use. Preferred excipients include sodium sulfate and leucine. The dry powders are typically manufactured by first processing the crystalline itraconazole to adjust the particle size using any number of techniques that are familiar to those of skill in the art (e.g., wet milling, jet milling). The crystalline itraconazole is processed in an antisolvent with polysorbate 80 to form a suspension. The stabilized suspension of crystalline itraconazole is then spray dried with the one or more additional excipients. The resulting dry particles comprise crystalline itraconazole dispersed throughout an excipient matrix with each dry particle having a homogenous composition.

In a particular embodiment, a dry powder of the present invention is made by starting with crystalline itraconazole, which is usually obtainable in a micro-crystalline size range. The particle size of the micro-crystalline itraconazole is reduced into the nano-crystalline size using any of a number of techniques familiar to those of skill in the art, including but not limited to, high-pressure homogenization, high-shear homogenization, jet-milling, pin milling, microfluidization, or wet milling (also known as ball milling, pearl milling or bead milling). Wet milling is often preferred, as it is able to achieve a wide range of particle size distributions, including those in the nanometer (<1 μm) size domain. What becomes especially important in the sub-micron size domain is the use of surface stabilizing components, such as surfactants (e.g., polysorbate 80, also called Tween 80). Polysorbate 80 enables the creation of submicron particles during milling and the formation of physically stable suspensions, as they sequester the many high energy surfaces created during milling preventing aggregation and sedimentation. Thus, the presence of the polysorbate 80 is important to spray drying homogenous micro-particles as the polysorbate 80 allows for the formation of a uniform and stable suspension ensuring compositional homogeneity across particles. The use of polysorbate 80 allows for formation of micro-suspensions or nano-suspensions. With the polysorbate 80, the nano-crystalline itraconazole particles are suspended in a stable colloidal suspension in the anti-solvent. The anti-solvent for the itraconazole can utilize water, or a combination of water and other miscible solvents such as alcohols or ketones as the continuous anti-solvent phase for the colloidal suspension. A spray drying feedstock may be prepared by dissolving the soluble components in a desired solvent(s) followed by dispersing the polysorbate 80-stabilized crystalline itraconazole nanosuspension in the resulting feedstock while mixing, although the process is not limited to this specific order of operations.

In some embodiments, variations of dry powders described herein are made by maintaining the amount of itraconazole, while reducing the amount of surfactant. In yet other embodiments, variations of the dry powders described herein are made by increasing the amount of itraconazole, while maintaining the original amount of surfactant.

Methods for analyzing the dry powders and/or respirable dry particles are found in the Exemplification section below.

### Therapeutic Use and Methods

The dry powders and/or respirable dry particles of the present invention are suitable for administration to the respiratory tract, for example to a subject in need thereof for the treatment of respiratory (e.g., pulmonary) diseases, such as cystic fibrosis, asthma, especially severe asthma, and severely immunocompromised patients. This treatment is especially useful in treating aspergillus infections. This treatment is also useful for treating fungal infections sensitive to itraconazole. Another aspect of the invention is treating allergic bronchopulmonary aspergillosis (ABPA), for example, in patients with pulmonary disease such as asthma or cystic fibrosis.

In other aspects, the invention is a method for the treatment, reduction in incidence or severity, or prevention of acute exacerbations caused by a fungal infection in the respiratory tract, such as an aspergillus infection. In another aspect, the invention is a method for the treatment, reduction in incidence or severity, or prevention of exacerbations caused by a fungal infection in the respiratory tract, such as an aspergillus infection. In another aspect, the invention is a method for the treatment, reduction in incidence or severity, or prevention of exacerbations caused by allergic bronchopulmonary aspergillosis (ABPA), for example, in patients with pulmonary disease such as asthma or cystic fibrosis.

In other aspects, the invention is a method for relieving the symptoms of a respiratory disease and/or a chronic pulmonary disease, such as cystic fibrosis, asthma, especially severe asthma and severely immunocompromised patients. In another aspect, the invention is a method for relieving the symptoms of allergic bronchopulmonary aspergillosis (ABPA) in these patient populations. In yet another aspect, the invention is a method for reducing inflammation, sparing the use of steroids, or reducing the need for steroidal treatment.

In other aspects, the invention is a method for improving lung function of a patient with a respiratory disease and/or a chronic pulmonary disease, such as such as cystic fibrosis, asthma, especially severe asthma and severely immunocompromised patients. In another aspect, the invention is a method for improving lung function of a patient with allergic bronchopulmonary aspergillosis (ABPA). In a further aspect, the invention is a method for prophylaxis or treatment of invasive fungal infections in an immunocompromised patient population.

The dry powders and/or respirable dry particles can be administered to the respiratory tract of a subject in need thereof using any suitable method, such as instillation techniques, and/or an inhalation device, such as a dry powder inhaler (DPI) or metered dose inhaler (MDI). A number of DPIs are available, such as, the inhalers disclosed is U.S. Pat. Nos. 4,995,385 and 4,069,819, Spinhaler® (Fisons, Loughborough, U.K.), Rotahalers®, Diskhaler® and Diskus® (GlaxoSmithKline, Research Triangle Technology Park, North Carolina), FlowCaps® (Hovione, Loures, Portugal), Inhalators® (Boehringer-Ingelheim, Germany), Aerolizer® (Novartis, Switzerland), high-resistance, ultrahigh-resistance and low-resistance RS01 (Plastiape, Italy) and others known to those skilled in the art.

The following scientific journal articles are incorporated by reference for their thorough overview of the following dry powder inhaler (DPI) configurations: 1) Single-dose Capsule DPI, 2) Multi-dose Blister DPI, and 3) Multi-dose Reservoir DPI. N. Islam, E. Gladki, “Dry powder inhalers (DPIs)—A review of device reliability and innovation”, International Journal of Pharmaceuticals, 360(2008):1-11. H. Chystyn, “Diskus Review”, International Journal of Clinical Practice, June 2007, 61, 6, 1022-1036. H. Steckel, B. Muller, “In vitro evaluation of dry powder inhalers I: drug deposition of commonly used devices”, International Journal of Pharmaceuticals, 154(1997):19-29. Some representative capsule-based DPI units are RS-01 (Plastiape, Italy), Turbospin® (PH&T, Italy), Brezhaler® (Novartis, Switzerland), Aerolizer (Novartis, Switzerland), Podhaler® (Novartis, Switzerland), HandiHaler® (Boehringer Ingelheim, Germany), AIR® (Civitas, Massachusetts), Dose One® (Dose One, Maine), and Eclipse® (Rhone Poulenc Rorer). Some representative unit dose DPIs are Conix® (3M, Minnesota), Cricket® (Mannkind, California), Dreamboat® (Mannkind, California), Occoris® (Team Consulting, Cambridge, UK), Solis® (Sandoz), Trivair® (Trimel Biopharma, Canada), Twincaps® (Hovione, Loures, Portugal). Some representative blister-based DPI units are Diskus® (GlaxoSmithKline (GSK), UK), Diskhaler® (GSK), Taper Dry® (3M, Minnesota), Gemini® (GSK), Twincer® (University of Groningen, Netherlands), Aspirair® (Vectura, UK), Acu-Breathe® (Respirics, Minnisota, USA), Exubra® (Novartis, Switzerland), Gyrohaler® (Vectura, UK), Omnihaler® (Vectura, UK), Microdose® (Microdose Therapeutix, USA), Multihaler® (Cipla, India) Prohaler® (Aptar), Technohaler® (Vectura, UK), and Xcelovair® (Mylan, Pennsylvania) . Some representative reservoir-based DPI units are Clickhaler® (Vectura), Next DPI® (Chiesi), Easyhaler® (Orion), Novolizer® (Meda), Pulmojet® (sanofi-aventis), Pulvinal® (Chiesi), Skyehaler® (Skyepharma), Duohaler® (Vectura), Taifun® (Akela), Flexhaler® (AstraZeneca, Sweden), Turbuhaler® (AstraZeneca, Sweden), and Twisthaler® (Merck), and others known to those skilled in the art.

Generally, inhalation devices (e.g., DPIs) are able to deliver a maximum amount of dry powder or dry particles in a single inhalation, which is related to the capacity of the blisters, capsules (e.g., size 000, 00, 0E, 0, 1, 2, 3 and 4, with respective volumetric capacities of 1.37 ml, 950 μl, 770 μl, 680 μl, 480 μl, 360 μl, 270 μl and 200 μl) or other means that contain the dry powders and/or respirable dry particles within the inhaler. Preferably, the blister has a volume of about 360 microliters or less, about 270 microliters or less, or more preferably, about 200 microliters or less, about 150 microliters or less, or about 100 microliters or less. Preferably, the capsule is a size 2 capsule, or a size 4 capsule. More preferably, the capsule is a size 3 capsule. Accordingly, delivery of a desired dose or effective amount may require two or more inhalations. Preferably, each dose that is administered to a subject in need thereof contains an effective amount of respirable dry particles or dry powder and is administered using no more than about 4 inhalations. For example, each dose of dry powder or respirable dry particles can be administered in a single inhalation or 2, 3, or 4 inhalations. The dry powders and/or respirable dry particles are preferably administered in a single, breath-activated step using a passive DPI. When this type of device is used, the energy of the subject's inhalation both disperses the respirable dry particles and draws them into the respiratory tract.

Dry powders and/or respirable dry particles suitable for use in the methods of the invention can travel through the upper airways (i.e., the oropharynx and larynx), the lower airways, which include the trachea followed by bifurcations into the bronchi and bronchioli, and through the terminal bronchioli which in turn divide into respiratory bronchioli leading then to the ultimate respiratory zone, the alveoli or the deep lung. In one embodiment of the invention, most of the mass of respirable dry particles deposit in the deep lung. In another embodiment of the invention, delivery is primarily to the central airways. In another embodiment, delivery is to the upper airways. In a preferred embodiment, most of the mass of the respirable dry particles deposit in the conducting airways.

If desired or indicated, the dry powders and respirable dry particles described herein can be administered with one or more other therapeutic agents. The other therapeutic agents can be administered by any suitable route, such as orally, parenterally (e.g., intravenous, intra-arterial, intramuscular, or subcutaneous injection), topically, by inhalation (e.g., intrabronchial, intranasal or oral inhalation, intranasal drops), rectally, vaginally, and the like. The respirable dry particles and dry powders can be administered before, substantially concurrently with, or subsequent to administration of the other therapeutic agent. Preferably, the dry powders and/or respirable dry particles and the other therapeutic agent are administered so as to provide substantial overlap of their pharmacologic activities.

The dry powders and respirable dry particles described herein are intended to be inhaled as such, and the present invention excludes the use of the dry powder formulation in making an extemporaneous dispersion. An extemporaneous dispersion is known by those skilled in the art as a preparation completed just before use, which means right before the administration of the drug to the patient. As used herein, the term “extemporaneous dispersion” refers to all of the cases in which the solution or suspension is not directly produced by the pharmaceutical industry and commercialized in a ready to be used form, but is prepared in a moment that follows the preparation of the dry solid composition, usually in a moment close to the administration to the patient.

### LIQUID FORMULATIONS

Liquid formulations for delivery with a pressurized metered dose inhaler (pMDI) or with a soft mist inhaler (SMI) can be prepared using any suitable method. For example, for use with a pMDI, a feedstock may be prepared inside a pressurized canister in which itraconazole in crystalline particulate form is suspended in a propellant such as a HFA propellant or a CFC propellant, optionally stabilized with a stabilizer such as polysorbate 80. The pressurized suspension may then be delivered into the respiratory tract of a patient by actuating the pMDI. Table 2 contains various embodiments for delivery of the itraconazole in crystalline particulate form by use of the pMDI. The nanoparticle solids concentration may vary from about 5%, about 10%, about 15%, about 20%, about 25%, about 30%, about 35%, about 40%, or about 50%. The dose volume of the pMDI may vary from about 20 uL to about 110 uL. The amount of itraconazole in the dose volume may be about 15%, 20%, 25%, 30% or 40%. The remainder of the volume may comprise propellant and optionally a surfactant. The pMDI delivery efficiency may be about 15%, 20%, 25%, 30% or 40%. Nominal doses of itraconazole in a pMDI may be varied from about 0.50 mg to about 12 mg. For example, the nominal dose may be about 2 mg, about 3 mg, about 4 mg, about 5 mg, about 6 mg, about 7 mg, about 8 mg, about 9 mg, about 10 mg or about 12 mg. The calculated delivery dose may range from about 0.1 mg to about 5 mg.

TABLE 2
Pressurized Metered Dose Inhaler (pMDI)

| | | Drug | | | |
|---|---|---|---|---|---|
| Nanoparticle | Dose | amount | pMDI | Nominal | Delivered |
| solids | volume | in dose | delivery | dose from | dose from |
| concentration | from pMDI | volume | efficiency | pMDI | pMDI |
| (%) | (uL) | (%) | (%) | (mg) | (mg) |
| 10 | 25 | 20 | 30 | 0.50 | 0.15 |
| 10 | 25 | 30 | 20 | 0.75 | 0.15 |
| 10 | 25 | 30 | 30 | 0.75 | 0.23 |
| 10 | 100 | 20 | 30 | 2.00 | 0.60 |
| 10 | 100 | 30 | 20 | 3.00 | 0.60 |
| 10 | 100 | 30 | 30 | 3.00 | 0.90 |
| 25 | 25 | 20 | 30 | 1.25 | 0.38 |
| 25 | 25 | 30 | 20 | 1.88 | 0.38 |
| 25 | 25 | 30 | 30 | 1.88 | 0.56 |
| 25 | 100 | 20 | 30 | 5.00 | 1.50 |
| 25 | 100 | 30 | 20 | 7.50 | 1.50 |
| 25 | 100 | 30 | 30 | 7.50 | 2.25 |
| 35 | 25 | 20 | 30 | 1.75 | 0.53 |
| 35 | 25 | 30 | 20 | 2.63 | 0.53 |
| 35 | 25 | 30 | 30 | 2.63 | 0.79 |
| 35 | 100 | 20 | 30 | 7.00 | 2.10 |
| 35 | 100 | 30 | 20 | 10.50 | 2.10 |
| 35 | 100 | 30 | 30 | 10.50 | 3.15 |

| | | |
|---|---|---|
| density of water: | 1 | g/mL |
| Unit conversion: | 1000 | mg/g |
| Unit conversion: | 1000 | uL/mL |

For use with an SMI, for example, a feedstock may be prepared in which itraconazole in crystalline particulate form is suspended in a solvent such as water in which the itraconazole is poorly soluble and stabilized with a stabilizer such as polysorbate 80. The suspension may be stored in a collapsible bag inside a cartridge which is loaded inside the device. A forced metered volume of suspension proceeds through a capillary tube into a micropump. Upon actuation of the SMI, a dose may be delivered to a patient. Table 3 contains various embodiments for delivery of the itraconazole in crystalline particulate form by use of the SMI. The nanoparticle solids concentration vary from about 5%, about 10%, about 15%, about 20%, about 25%, about 30%, about 35%, about 40%, or about 50%. The dose volume of the SMI may vary from about 10 uL to about 25 uL. The formulation may comprise itraconazole in crystalline particulate form and surfactant. The SMI delivery efficiency may be about 65%, 70%, 75%, 80%, or 85%. Nominal doses of itraconazole in a pMDI may vary from about 1.0 mg to about 8 mg. For example, the nominal dose may be about 2 mg, about 3 mg, about 4 mg, about 5 mg, about 6 mg, about 7 mg, or about 8 mg. The calculated delivery dose may range from about 0.5 mg to about 5 mg.

TABLE 3
Soft Mist Inhaler (SMI)

| Nanoparticle | Dose | pMDI | Nominal | Delivered |
|---|---|---|---|---|
| solids | volume | delivery | dose from | dose from |
| concentration | from SMI | efficiency | pMDI | pMDI |
| (%) | (uL) | (%) | (mg) | (mg) |
| 10 | 15 | 75 | 1.50 | 1.13 |
| 25 | 15 | 75 | 3.75 | 2.81 |
| 35 | 15 | 75 | 5.25 | 3.94 |

| | | |
|---|---|---|
| density of water: | 1 | g/mL |
| Unit conversion: | 1000 | mg/g |
| Unit conversion: | 1000 | uL/mL |

### EXEMPLIFICATION

Materials used in the following Examples and their sources are listed below. Sodium chloride, sodium sulfate, polysorbate 80, ammonium hydroxide, mannitol, magnesium lactate, and L-leucine were obtained from Sigma-Aldrich Co. (St. Louis, Mo.), Spectrum Chemicals (Gardena, Calif.), Applichem (Maryland Heights, Mo.), Alfa Aesar (Tewksbury, Mass.), Thermo Fisher (Waltham, Mass.), Croda Chemicals (East Yorkshire, United Kingdom) or Merck (Darmstadt, Germany). Itraconazole was obtained from Neuland (Princeton, N.J.) or SMS Pharmaceutical ltd (Telengana State, India). Ultrapure (Type II ASTM) water was from a water purification system (Millipore Corp., Billerica, Mass.), or equivalent.

Methods:

Geometric of Volume Diameter of Suspensions. Volume median diameter (x50 or Dv50), which may also be referred to as volume median geometric diameter (VMGD), of the active agent suspensions was determined using a laser diffraction technique. The equipment consisted of a Horiba LA-950 instrument outfitted with an automated recirculation system for sample handling and removal or a fixed-volume sample cuvette. The sample to a dispersion media, consisting of either deionized water or deionized water with less than 0.5% of a surfactant such as polysorbate 80 or sodium dodecyl sulfate. Ultrasonic energy can be applied to aid in dispersion of the suspension. When the laser transmission was in the correct range, the sample was sonicated for 60 seconds at a setting of 5. The sample was then measured and the particle size distribution reported.

Geometric or Volume Diameter of Dry Powders. Volume median diameter (x50 or Dv50), which may also be referred to as volume median geometric diameter (VMGD), of the dry powder formulations was determined using a laser diffraction technique. The equipment consisted of a HELOS diffractometer and a RODOS dry powder disperser (Sympatec, Inc., Princeton, N.J.). The RODOS disperser applies a shear force to a sample of particles, controlled by the regulator pressure (typically set at 1.0 bar with maximum orifice ring pressure) of the incoming compressed dry air. The pressure settings may be varied to vary the amount of energy used to disperse the powder. For example, the dispersion energy may be modulated by changing the regulator pressure from 0.2 bar to 4.0 bar. Powder sample is dispensed from a microspatula into the RODOS funnel. The dispersed particles travel through a laser beam where the resulting diffracted light pattern produced is collected, typically using an R1 lens, by a series of detectors. The ensemble diffraction pattern is then translated into a volume-based particle size distribution using the Fraunhofer diffraction model, on the basis that smaller particles diffract light at larger angles. Using this method, the span of the distribution was also determined per the formula (Dv[90]−Dv[10)/Dv[50]. The span value gives a relative indication of the polydispersity of the particle size distribution.

Aerodynamic Performance via Andersen Cascade Impactor The aerodynamic properties of the powders dispersed from an inhaler device were assessed with an Mk-II 1 ACFM Andersen Cascade Impactor (Copley Scientific Limited, Nottingham, UK) (ACI). The ACI instrument was run in controlled environmental conditions of 18 to 25° C. and relative humidity (RH) between 25 and 35%. The instrument consists of eight stages that separate aerosol particles based on inertial impaction. At each stage, the aerosol stream passes through a set of nozzles and impinges on a corresponding impaction plate. Particles having small enough inertia will continue with the aerosol stream to the next stage, while the remaining particles will impact upon the plate. At each successive stage, the aerosol passes through nozzles at a higher velocity and aerodynamically smaller particles are collected on the plate. After the aerosol passes through the final stage, a filter collects the smallest particles that remain, called the “final collection filter”. Gravimetric and/or chemical analyses can then be performed to determine the particle size distribution. A short stack cascade impactor, also referred to as a collapsed cascade impactor, is also utilized to allow for reduced labor time to evaluate two aerodynamic particle size cut-points. With this collapsed cascade impactor, stages are eliminated except those required to establish fine and coarse particle fractions. The impaction techniques utilized allowed for the collection of two or eight separate powder fractions. The capsules (HPMC, Size 3; Capsugel Vcaps, Peapack, N.J.) were filled with powder to a specific weight and placed in a hand-held, breath-activated dry powder inhaler (DPI) device, the high resistance RS01 DPI or the ultra-high resistance UHR2 DPI (both by Plastiape, Osnago, Italy). The capsule was punctured and the powder was drawn through the cascade impactor operated at a flow rate of 60.0 L/min for 2.0 s. At this flowrate, the calibrated cut-off diameters for the eight stages are 8.6, 6.5, 4.4, 3.3, 2.0, 1.1, 0.5 and 0.3 microns and for the two stages used with the short stack cascade impactor, based on the Andersen Cascade Impactor, the cut-off diameters are 5.6 microns and 3.4 microns. The fractions were collected by placing filters in the apparatus and determining the amount of powder that impinged on them by gravimetric measurements or chemical measurements on an HPLC.

Aerodynamic Performance via Next Generation Impactor. The aerodynamic properties of the powders dispersed from an inhaler device were assessed with a Next Generation Impactor (Copley Scientific Limited, Nottingham, UK) (NGI). For measurements utilizing the NGI, the NGI instrument was run in controlled environmental conditions of 18 to 25° C. and relative humidity (RH) between 25 and 35%. The instrument consists of seven stages that separate aerosol particles based on inertial impaction and can be operated at a variety of air flow rates. At each stage, the aerosol stream passes through a set of nozzles and impinges on a corresponding impaction surface. Particles having small enough inertia will continue with the aerosol stream to the next stage, while the remaining particles will impact upon the surface. At each successive stage, the aerosol passes through nozzles at a higher velocity and aerodynamically smaller particles are collected on the plate. After the aerosol passes through the final stage, a micro-orifice collector collects the smallest particles that remain. Gravimetric and/or chemical analyses can then be performed to determine the particle size distribution. The capsules (HPMC, Size 3; Capsugel Vcaps, Peapack, N.J.) were filled with powder to a specific weight and placed in a hand-held, breath-activated dry powder inhaler (DPI) device, the high resistance RS01 DPI or the ultra-high resistance RS01 DPI (both by Plastiape, Osnago, Italy). The capsule was punctured and the powder was drawn through the cascade impactor operated at a specified flow rate for 2.0 Liters of inhaled air. At the specified flow rate, the cut-off diameters for the stages were calculated. The fractions were collected by placing wetted filters in the apparatus and determining the amount of powder that impinged on them by chemical measurements on an HPLC.

Fine Particle Dose The fine particle dose indicates the mass of one or more therapeutics in a specific size range and can be used to predict the mass which will reach a certain region in the respiratory tract. The fine particle dose can be measured gravimetrically or chemically via either an ACI or NGI. If measured gravimetrically, since the dry particles are assumed to be homogenous, the mass of the powder on each stage and collection filter can be multiplied by the fraction of therapeutic agent in the formulation to determine the mass of therapeutic. If measured chemically, the powder from each stage or filter is collected, separated, and assayed for example on an HPLC to determine the content of the therapeutic. The cumulative mass deposited on each of the stages at the specified flow rate is calculated and the cumulative mass corresponding to a 5.0 micrometer diameter particle is interpolated. This cumulative mass for a single dose of powder, contained in one or more capsules, actuated into the impactor is equal to the fine particle dose less than 5.0 microns (FPD<5.0 microns).

Mass Median Aerodynamic Diameter. Mass median aerodynamic diameter (MMAD) was determined using the information obtained by the Andersen Cascade Impactor (ACI). The cumulative mass under the stage cut-off diameter is calculated for each stage and normalized by the recovered dose of powder. The MMAD of the powder is then calculated by linear interpolation of the stage cut-off diameters that bracket the 50th percentile. An alternative method of measuring the MMAD is with the Next Generation Impactor (NGI). Like the ACI, the MMAD is calculated with the cumulative mass under the stage cut-off diameter is calculated for each stage and normalized by the recovered dose of powder. The MMAD of the powder is then calculated by linear interpolation of the stage cut-off diameters that bracket the 50th percentile.

Emitted Geometric or Volume Diameter. The volume median diameter (Dv50) of the powder after it is emitted from a dry powder inhaler, which may also be referred to as volume median geometric diameter (VMGD), was determined using a laser diffraction technique via the Spraytec diffractometer (Malvern, Inc.). Powder was filled into size 3 capsules (V-Caps, Capsugel) and placed in a capsule based dry powder inhaler (RS01 Model 7 High resistance, Plastiape, Italy), or DPI, and the DPI sealed inside a cylinder. The cylinder was connected to a positive pressure air source with steady air flow through the system measured with a mass flow meter and its duration controlled with a timer controlled solenoid valve. The exit of the dry powder inhaler was exposed to room pressure and the resulting aerosol jet passed through the laser of the diffraction particle sizer (Spraytec) in its open bench configuration before being captured by a vacuum extractor. The steady air flow rate through the system was initiated using the solenoid valve. A steady air flow rate was drawn through the DPI typically at 60 L/min for a set duration, typically of 2 seconds. Alternatively, the air flow rate drawn through the DPI was sometimes run at 15 L/min, 20 L/min, or 30 L/min. The resulting geometric particle size distribution of the aerosol was calculated from the software based on the measured scatter pattern on the photodetectors with samples typically taken at 1000 Hz for the duration of the inhalation. The Dv50, GSD, FPF<5.0 μm measured were then averaged over the duration of the inhalation.

Emitted Dose (ED) refers to the mass of therapeutic which exits a suitable inhaler device after a firing or dispersion event. The ED is determined using a method based on USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, Md., 13th Revision, 222-225, 2007. Contents of capsules are dispersed using either the RS01 HR inhaler at a pressure drop of 4 kPa and a typical flow rate of 60 LPM or the UHR2 RS01 at a pressure drop of 4 kPa and a typical flow rate of 39 LPM. The emitted powder is collected on a filter in a filter holder sampling apparatus. The sampling apparatus is rinsed with a suitable solvent such as water and analyzed using an HPLC method. For gravimetric analysis a shorter length filter holder sampling apparatus is used to reduce deposition in the apparatus and the filter is weighed before and after to determine the mass of powder delivered from the DPI to the filter. The emitted dose of therapeutic is then calculated based on the content of therapeutic in the delivered powder. Emitted dose can be reported as the mass of therapeutic delivered from the DPI or as a percentage of the filled dose. ED can also be calculated from the results generated by Nex Generation Impactor (NGI) experiments, through summation of all of the drug or powder assayed from the mouthpiece adapter, NGI induction port, and all of the stages within the NGI. The results generated through ED testing per USP 601 and the results generated via the NGI are typically in good agreement.

Thermogravimetric Analysis: Thermogravimetric analysis (TGA) was performed using either the Q500 model or the Discovery model thermogravimetric analyzer (TA Instruments, New Castle, Del.). The samples were either placed into an open aluminum DSC pan or a sealed aluminum DSC pan that was then automatically punched open prior to the time of test. Tare weights were previously recorded by the instrument. The following method was employed: Ramp 5.00° C./min from ambient (˜35° C). to 200° C. The weight loss was reported as a function of temperature up to 140° C. TGA allows for the calculation of the content of volatile compounds within the dry powder. When utilizing processes with water alone, or water in conjunction with volatile solvents, the weight loss via TGA is a good estimate of water content.

X-Ray Powder Diffraction: The crystalline character of the formulations was assessed via powder X-ray diffraction (PXRD). A 20-30 mg sample of material is analyzed in a powder X-ray diffractometer (D8 Discover with LINXEYE detector; Bruker Corporation, Billerica, Mass. or equivalent) using a Cu X-ray tube with 1.5418 A at a data accumulation time 1.2 second/step over a scan range of 5 to 45° 2θ and a step size of 0.02°θ.

Itraconazole Content/Purity using HPLC. A high performance liquid chromatography (HPLC) method utilizing a reverse phase C18 column coupled to an ultraviolet (UV) detector has been developed for the identification, bulk content, assay, CUPMD and impurities analysis of itraconazole formulations. The reverse phase column is equilibrated to 30° C. and the autosampler is set to 5° C. The mobile phases, 20 mM sodium phosphate monobasic at a pH of 2.0 (mobile phase A) and acetonitrile (mobile phase B) are used in a gradient elution from a ratio of 59:41 (A:B) to 5:95 (A:B), over the course of a 19.5 minute run time. Detection is by UV at 258 nm and the injection volume is 10 μL. Itraconazole content in powders are quantified relative to a standard curve.

Identification of known impurities A, B, C, D, E, F and G (shown in monograph Ph. Eur. 01/2011:1335) is confirmed by comparing the retention time of the impurity peaks in the itraconazole formulation samples to that of the itraconazole USP impurity mix reference standard spiked with impurity A. Unknown impurities are identified and quantified by relative retention time to that of the itraconazole main peak and with area above the limit of detection (LOD). All impurities are measured by area percent, with respect to the itraconazole peak.

Particle Size Reduction. The particle size distribution of the crystalline active agent can be modulated using a number of techniques familiar to those of skill in the art, including but not limited to, high-pressure homogenization, high-shear homogenization, jet-milling, pin milling, microfluidization, or wet milling (also known as ball milling, pearl milling or bead milling). Wet milling is often preferred, as it is able to achieve a wide range of particle size distributions, including those in the nanometer (<1 μm) size domain.

Particle Size Reduction using Low Energy Wet Milling. One technique for reducing the particle size of the active agent was via low energy wet milling, (also known as roller milling, or jar milling). Suspensions of the active agent were prepared in an anti-solvent, which can be water, or any solvent in which the active agent is not appreciably soluble. Stabilizers, which can be, but are not limited to, non-ionic surfactants or amphiphilic polymers, are then added to the suspension along with milling media, which can be, but are not limited to, spherical with high wear resistance and in the size range from 0.03 to 0.70 millimeters in diameter. The vessels containing the suspensions are then rotated using a jar mill (US Stoneware, East Palestine, Ohio USA) while taking samples periodically to assess particle size (LA-950, HORIBA, Kyoto, Japan). When the particle size is sufficiently reduced, or when a particle size minimum is reached, the suspension is strained through a sieve to remove the milling media, and the product recovered.

Particle Size Reduction using High Energy Wet Milling. Another technique for reducing the particle size of the active agent was via high-energy wet milling using a rotor-stator, or agitated media mill. Suspensions of the active agent were prepared in an anti-solvent, which can be water, or any solvent in which the active agent is not appreciably soluble. Stabilizers, which can be, but are not limited to, non-ionic surfactants or amphiphilic polymers, are then added to the suspension along with milling media, which can be, but are not limited to, spherical with high wear resistance and in the size range from 0.03 to 0.70 millimeters in diameter. The suspensions are then charged into the mill, which can be operated in either batch or recirculation mode. The process consists of the suspension and milling media being agitated within the milling chamber, which increases the energy input to the system and accelerates the particle size reduction process. The milling chamber and recirculation vessel are jacketed and actively cooled to avoid temperature increases in the product. The agitation rate and recirculation rate of the suspension are controlled during the process. Samples are taken periodically to assess particle size (LA-950, HORIBA, Kyoto, Japan). When the particle size is sufficiently reduced, or when a particle size minimum is reached, the suspension is discharged from the mill.

Particle Size Reduction using Microfluidization. Another technique for reducing the particle size distribution of the active agent was via Microfluidization. Microfluidizer-based processing is a high-shear wet-processing unit operation utilized for particle size reduction of liquids and solids. The unit can be configured with various interaction chambers, which are cylindrical modules with specific orifice and channel designs through which fluid is passed at high pressures to control shear rates. Product enters the unit via the inlet reservoir and is forced into the fixed-geometry interaction chamber at speeds up to 400 m/sec by a high-pressure pump. It is then effectively cooled, if required, and collected in the output reservoir. The process can be repeated as necessary (e.g. multiple “passes”) to achieve the particle size targets. Particle size of the active agent is monitored periodically via laser diffraction (LA-950, HORIBA, Kyoto, Japan). When the particle size is sufficiently reduced, or when a particle size minimum is reached, the suspension is recovered from the unit.

Particle Size Reduction using Jet Milling Another technique for reducing the particle size distribution of the active agent was via jet milling. Jet mills utilize fluid energy (compressed air or gas) to grind and classify, in a single chamber with no moving parts. Activated by high pressure air, the particles are accelerated into a high speed rotation in a shallow grinding chamber. As the particles impact on one another their size is reduced. Centrifugal force holds larger particles in the grinding rotation area until they have achieved the desired fine particle size. Centripetal force drags the desired particles towards the static classifier where they are allowed to exit upon achieving the correct particle size. The final particle size is controlled by varying the rate of the feed and propellant pressure.

Liquid Feedstock Preparation for Spray Drying. Spray drying homogenous particles requires that the ingredients of interest be solubilized in solution or suspended in a uniform and stable suspension. The feedstock can utilize water, or a combination of water and other miscible solvents such as alcohols or ketones, as the solvent in the case of solutions, or as the continuous phase in the case of suspensions. Feedstocks of the various formulations were prepared by dissolving the soluble components in the desired solvent(s) followed by dispersing the surfactant-stabilized active agent-containing suspension in the resulting solution while mixing, although the process is not limited to this specific order of operations.

Spray Drying Using Niro Spray Dryer. Dry powders were produced by spray drying utilizing a Niro Mobile Minor spray dryer (GEA Process Engineering Inc., Columbia, Md.) with powder collection from a cyclone, a product filter or both. Atomization of the liquid feed was performed using a co-current two-fluid nozzle either from Niro (GEA Process Engineering Inc., Columbia, Md.) or a Spraying Systems (Carol Stream, Ill.) 1/4 J two-fluid nozzle with gas cap 67147 and fluid cap 2850SS, although other two-fluid nozzle setups are also possible. In some embodiments, the two-fluid nozzle can be in an internal mixing setup or an external mixing setup. Additional atomization techniques include rotary atomization or a pressure nozzle. The liquid feed was fed using gear pumps (Cole-Parmer Instrument Company, Vernon Hills, Ill.) directly into the two-fluid nozzle or into a static mixer (Charles Ross & Son Company, Hauppauge, N.Y.) immediately before introduction into the two-fluid nozzle. An additional liquid feed technique includes feeding from a pressurized vessel. Nitrogen or air may be used as the drying gas, provided that moisture in the air is at least partially removed before its use. Pressurized nitrogen or air can be used as the atomization gas feed to the two-fluid nozzle. The drying gas inlet temperature can range from 70° C. to 300° C. and outlet temperature from 30° C. to 120° C. with a liquid feedstock rate of 10 mL/min to 100 mL/min. The gas supplying the two-fluid atomizer can vary depending on nozzle selection and for the Niro co-current two-fluid nozzle can range from 5 kg/hr to 50 kg/hr or for the Spraying Systems 1/4J two-fluid nozzle can range from 30 g/min to 150 g/min. The atomization gas rate can be set to achieve a certain gas to liquid mass ratio, which directly affects the droplet size created. The pressure inside the drying drum can range from +3 ″WC to −6 ″WC. Spray dried powders can be collected in a container at the outlet of the cyclone, onto a cartridge or baghouse filter, or from both a cyclone and a cartridge or baghouse filter.

Spray Drying Using Büchi Spray Dryer. Dry powders were prepared by spray drying on a Büchi B-290 Mini Spray Dryer (BÜCHI Labortechnik AG, Flawil, Switzerland) with powder collection from either a standard or High Performance cyclone. The system was run either with air or nitrogen as the drying and atomization gas in open-loop (single pass) mode. When run using air, the system used the Büchi B-296 dehumidifier to ensure stable temperature and humidity of the air used to spray dry. Furthermore, when the relative humidity in the room exceeded 30% RH, an external LG dehumidifier (model 49007903, LG Electronics, Englewood Cliffs, N.J.) was run constantly. When run using nitrogen, a pressurized source of nitrogen was used. Furthermore, the aspirator of the system was adjusted to maintain the system pressure at −2.0″ water column. Atomization of the liquid feed utilized a Büchi two-fluid nozzle with a 1.5 mm diameter or a Schlick 970-0 atomizer with a 0.5 mm liquid insert (Düsen-Schlick GmbH, Coburg, Germany). Inlet temperature of the process gas can range from 100° C. to 220° C. and outlet temperature from 30° C. to 120° C. with a liquid feedstock flowrate of 3 mL/min to 10 mL/min. The two-fluid atomizing gas ranges from 25 mm to 45 mm (300 LPH to 530 LPH) for the Büchi two-fluid nozzle and for the Schlick atomizer an atomizing air pressure of upwards of 0.3 bar. The aspirator rate ranges from 50% to 100%.

Stability Assessment: The physicochemical stability and aerosol performance of select formulations were assessed at 2-8° C., 25° C./60% RH, and when material quantities permitted, 40° C./75% RH as detailed in the International Conference on Harmonisation (ICH) Q1 guidance. Stability samples were stored in calibrated chambers (Darwin Chambers Company Models PH024 and PH074, St. Louis. Mo.). Bulk powder samples were weighed into amber glass vials, sealed under 30% RH, and induction-sealed in aluminum pouches (Drishield 3000, 3M, St. Paul, Minn.) with silica desiccant (2.0 g, Multisorb Technologies, Buffalo, N.Y.). Additionally, to assess the stability of the formulations in capsules, the target mass of powder was weighed by hand into a size 3, HPMC capsule (Capsugel Vcaps, Peapack, N.J.)) at 30% RH or less. Filled capsules were then aliquoted into high-density polyethylene (HDPE) bottles and induction sealed in aluminum pouches with silica desiccant.

### A. Example 1. Dry Powder Formulations of Itraconazole in Crystalline Particulate Form at Varying Drug Loads Powder Preparation

The nanocrystalline itraconazole for Formulations XI-XVI was prepared as a suspension comprising 35.0 wt % itraconazole (SMS Pharma lot ITZ-0715005) and 2.92 wt % polysorbate 80, comprising a 12:1 ratio (wt:wt) of itraconazole to polysorbate 80. The polysorbate 80 was dissolved in 62.1% deionized water via magnetic stir bar, then the itraconazole was added and suspended by stirring with a magnetic stir bar. Once all of the itraconazole was suspended, the formulation was processed on the Netzsch MiniCer using 0.2 mm grinding media (TOSOH, Tokyo, Japan) with 90% chamber fill. The following conditions were used to manufacture the itraconazole suspension. The mill speed was 3000 RPM, the inlet pump flow rate was 220 mL/min, the recirculating chiller was 10° C., and the run time was 37 minutes. The final median particle size (Dv(50)) of the milled suspension was 141 nm.

Feedstock suspensions were prepared and used to manufacture dry powders comprising itraconazole in crystalline particulate form and additional excipients. Drug loads of 50, 60, 70 and 80 wt % itraconazole, on a dry basis, were targeted. The feedstock suspensions that were used to spray dry particles were made as follows. The required quantity of water was weighed into a suitably sized glass vessel. The excipients were added to the water and the solution was allowed to stir until visually clear. The itraconazole-containing nano-suspension was then added to the excipient solution and stirred until visually homogenous. The feedstocks were then spray-dried. Feedstocks were stirred while spray dried. Table 4 lists the components of the feedstocks used in preparation of the dry powders.

TABLE 4
Feedstock compositions for Formulations XI-XVI

| | | | Polysorbate | Sodium | | |
|---|---|---|---|---|---|---|
| | Water | Itraconazole | 80 | sulfate | Leucine | Total mass |
| Formulation | (g) | (g) | (g) | (g) | (g) | (g) |
| XI | 67.891 | 1.050 | 0.087 | 0.749 | 0.216 | 69.992 |
| XII | 67.918 | 1.260 | 0.105 | 0.573 | 0.162 | 70.019 |
| XIII | 67.911 | 1.470 | 0.122 | 0.395 | 0.114 | 70.012 |
| XIV | 67.910 | 1.680 | 0.140 | 0.218 | 0.062 | 70.010 |
| XV | 67.896 | 1.680 | 0.140 | 0.139 | 0.141 | 69.996 |
| XVI | 67.897 | 1.680 | 0.140 | 0.062 | 0.218 | 69.996 |

Dry powders of Formulations XI-XVI were manufactured from the corresponding feedstocks in Table 4 by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Schlick 970-1 nozzle. The aspirator of the system was adjusted to maintain the system pressure at −2.0″ water column.

The following spray drying conditions were followed to manufacture the dry powders. The liquid feedstock solids concentration was 30 g/kg, the drying gas flowrate was 17.0 kg/hr, the atomization gas flowrate was 19.6 g/min, and the liquid feedstock flowrate was 3.0 mL/min. The process gas inlet temperature was varied to keep the outlet temperature constant at 65° C. The resulting dry powder formulations are reported in Table 5.

TABLE 5
Formulation XI-XVI compositions, dry basis

| Formulation | Dry Powder Composition (w/w), dry basis |
|---|---|
| XI | 50.0% itraconazole, 35.7% sodium sulfate, 10.2% |
| | leucine, 4.16% polysorbate 80 |
| XII | 60.0% itraconazole, 27.2% sodium sulfate, 7.78% |
| | leucine, 5.00% polysorbate 80 |
| XIII | 70.0% itraconazole, 18.8% sodium sulfate, 5.37% |
| | leucine, 5.83% polysorbate 80 |
| XIV | 80.0% itraconazole, 10.4% sodium sulfate, 2.96% |
| | leucine, 6.67% polysorbate 80 |
| XV | 80.0% itraconazole, 6.67% sodium sulfate, 6.66% |
| | leucine, 6.67% polysorbate 80 |
| XVI | 80.0% itraconazole, 2.96% sodium sulfate, 10.4% |
| | leucine, 6.67% polysorbate 80 |

### B. Powder Characterization

The bulk particle size characteristics for the six formulations are found in Table 6. The 1 bar/4 bar dispersibility ratio less than 1.1 and 0.5 bar/4 bar dispersibility ratio less than 1.25 for Formulations XI-XVI indicate that they are relatively independent of dispersion energy, a desirable characteristic which allows similar particle dispersion across a range of dispersion energies.

TABLE 6
Formulation XI-XVI Bulk particle size

| | 1 bar | | |
|---|---|---|---|
| | Dv[50] | 1 bar:4 bar | 0.5 bar:4 bar |
| Formulation | (μm) | Dv[50] ratio | Dv[50] ratio |
| XI | 2.47 | 1.01 | 1.11 |
| XII | 2.73 | 1.08 | 1.15 |
| XIII | 2.65 | 1.08 | 1.20 |
| XIV | 2.61 | 1.04 | 1.14 |
| XV | 2.85 | 1.00 | 1.00 |
| XVI | 2.40 | 1.02 | 1.15 |

The weight loss of Formulations XI-XVI was measured via TGA and is detailed in Table 7.

TABLE 7
Formulation XI-XVI weight loss via TGA

| Formulation | Weight loss via TGA (%) |
|---|---|
| XI | 0.23 |
| XII | 0.69 |
| XIII | 0.18 |
| XIV | 0.33 |
| XV | 0.64 |
| XVI | 0.37 |

The aerodynamic particle size, fine particle fractions, and fine particle doses measured and/or calculated with a Next Generation Impactor (NGI) for Formulations XI, XIV and XVI are reported in Table 8. The fine particle doses for all formulations indicate a high percentage of the nominal dose which is filled into the capsule reaches the impactor stages (>45%) and so would be predicted to be delivered to the lungs. The MMADs of all formulations were <3.5 μm microns, indicating deposition in the central and conducting airways.

TABLE 8
Formulation XI-XVI aPSD via NGI

| | MMAD | FPD < 5 μm |
|---|---|---|
| Formulation | (μm) | (% nominal dose) |
| XI | 3.12 | 53.6 |
| XIV | 3.18 | 48.8 |
| XVI | 3.31 | 54.8 |

### Example 2. Improved Aerosol Performance of 12:1 Itraconazole:PS80 Ratio Formulations Versus 10:1 Formulations

### A. Powder Preparation

Three formulations (XXVIII, XXIX, XXX) with the same itraconazole load and excipient ratio as Formulations XI, XIV and XVI, but with a 10:1 itraconazole:PS80 ratio, were manufactured to assess any performance differences as a function of itraconazole:PS80 ratio. The composition of these 10:1 itraconazole:PS80 formulations are presented in Table 9, alongside their 12:1 counterparts.

TABLE 9
Composition of Formulations XXVII-XXX, dry basis

| | | Sodium | | Polysorbate | | Sodium |
|---|---|---|---|---|---|---|
| | Itraconazole | Sulfate | Leucine | 80 | Itraconazole:PS | Sulfate:Leucine |
| Formulation | (wt %) | (wt %) | (wt %) | (wt %) | 80 ratio | Ratio |
| XXVIII | 50.0 | 35.0 | 10.0 | 5.00 | 10:1 | 3.5:1 |
| XXIX | 80.0 | 9.33 | 2.67 | 8.00 | 10:1 | 3.5:1 |
| XXX | 80.0 | 2.67 | 9.33 | 8.00 | 10:1 | 1:3.5 |
| XI | 50.0 | 35.7 | 10.2 | 4.16 | 12:1 | 3.5:1 |
| XIV | 80.0 | 10.4 | 2.96 | 6.67 | 12:1 | 3.5:1 |
| XVI | 80.0 | 2.96 | 10.4 | 6.67 | 12:1 | 1:3.5 |

The nanocrystalline itraconazole for Formulations XXVIII-XXX was prepared as a suspension comprising 35.0 wt % itraconazole (SMS Pharma lot ITZ-0715005) and 3.50 wt % polysorbate 80, comprising a 10:1 ratio (wt:wt) of itraconazole to polysorbate 80. The polysorbate 80 was dissolved in deionized water via magnetic stir bar, then the itraconazole was added and suspended by stirring with a magnetic stir bar. Once all of the itraconazole was suspended, the formulation was processed on the Netzsch MiniCer using 0.2 mm grinding media (TOSOH, Tokyo, Japan) with 90% chamber fill. The following conditions were used to manufacture the itraconazole suspension. The mill speed was 3000 RPM, the inlet pump flow rate was 216 mL/min, the recirculating chiller was 10° C., and the run time was 37 minutes. The final median particle size (Dv(50)) of the milled suspension was 135 nm.

Feedstock suspensions were prepared and used to manufacture dry powders comprising itraconazole in crystalline particulate form and additional excipients. Drug loads of 50 and 80 wt % itraconazole, on a dry basis, were targeted. The feedstock suspensions that were used to spray dry particles were made as follows. The required quantity of water was weighed into a suitably sized glass vessel. The excipients were added to the water and the solution was allowed to stir until visually clear. The itraconazole-containing nano-suspension was then added to the excipient solution and stirred until visually homogenous. The feedstocks were then spray-dried. Feedstocks were stirred while spray dried. Table 10 lists the components of the feedstocks used in preparation of the dry powders.

TABLE 10
Feedstock compositions for Formulations XXVIII-XXX

| | | | Polysorbate | Sodium | | |
|---|---|---|---|---|---|---|
| | Water | Itraconazole | 80 | sulfate | Leucine | Total mass |
| Formulation | (g) | (g) | (g) | (g) | (g) | (g) |
| XXVIII | 67.820 | 1.125 | 0.113 | 0.735 | 0.211 | 70.004 |
| XXIX | 67.901 | 1.680 | 0.168 | 0.197 | 0.056 | 70.002 |
| XXX | 67.907 | 1.680 | 0.168 | 0.056 | 0.196 | 70.007 |

Dry powders of Formulations XXVIII-XXX were manufactured from the corresponding feedstocks in Table 10 by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Schlick 970-1 nozzle. The aspirator of the system was adjusted to maintain the system pressure at −2.0″ water column.

The following spray drying conditions were used to manufacture the dry powders. The liquid feedstock solids concentration was 30 g/kg, the drying gas flowrate was 17.0 kg/hr, the atomization gas flowrate was 19.6 g/min, and the liquid feedstock flowrate was 3.0 mL/min. The process gas inlet temperature was varied to keep the outlet temperature constant at 65° C. The resulting dry powder formulations are provided in Table 9.

### B. Powder Characterization.

The bulk particle size characteristics for the three formulations are found in Table 11. The 1 bar/4 bar dispersibility ratio less than 1.1 and 0.5 bar/4 bar dispersibility ratio less than 1.25 for Formulations XXVIII-XXX indicate that they are relatively independent of dispersion energy, a desirable characteristic which allows similar particle dispersion across a range of dispersion energies.

TABLE 11
Formulation XXVIII-XXX Bulk particle size

| | 1 bar | | |
|---|---|---|---|
| | Dv[50] | 1 bar:4 bar | 0.5 bar:4 bar |
| Formulation | (μm) | Dv[50] ratio | Dv[50] ratio |
| XXVIII | 2.65 | 0.97 | 1.10 |
| XXIX | 3.21 | 1.08 | 1.08 |
| XXX | 2.60 | 1.07 | 1.14 |

The weight loss of Formulations XXVII-XXX was measured via TGA and is detailed in Table 12.

TABLE 12
Formulation XXVIII-XXX weight loss via TGA

| Formulation | Weight loss via TGA (%) |
|---|---|
| XXVIII | 0.80 |
| XXIX | 0.42 |
| XXX | 0.23 |

The aerodynamic particle size, fine particle fractions, and fine particle doses measured and/or calculated with a Next Generation Impactor (NGI) for Formulations XXVIII, XXIX and XXX are reported in Table 13. The fine particle doses for all formulations indicate a high percentage of the nominal dose which is filled into the capsule reaches the impactor stages and so would be predicted to be delivered to the lungs. The MMADs of all formulations were between 3.23 μm and 3.47 μm, indicating deposition in the central and conducting airways.

TABLE 13
Formulation XXVIII-XXX aPSD via NGI

| | MMAD | FPD < 5 μm |
|---|---|---|
| Formulation | (μm) | (% nominal dose) |
| XXVIII | 3.43 | 47.6 |
| XXIX | 3.47 | 42.4 |
| XXX | 3.23 | 50.6 |

### C. Comparison of 12:1 Itraconazole:PS80 and 10:1 Itraconazole:PS80 Formulations

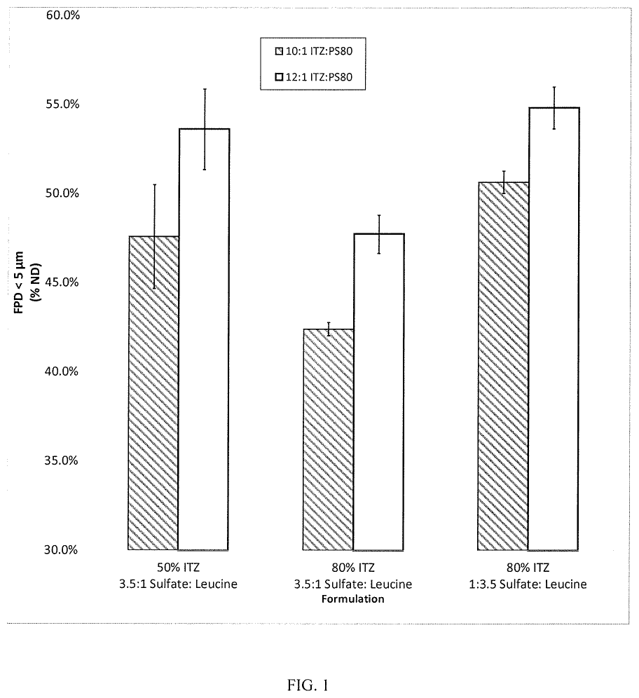

Comparison of the aerosol performance of the 12:1 itraconazole:PS80 formulations to the 10:1 itraconazole:PS80 formulations is presented in Table 14 and illustrated in FIG. 1. As shown, the 12:1 itraconazole:PS80 ratio shows an increased fine particle dose <5.0 μm at both 50% and 80% itraconazole load, and across a range of excipient ratios (3.5:1 to 1:3.5). The increase from Formulation XXVIII to Formulation XI was 6.0%. The increase from Formulation XXIX to Formulation XIV was 6.4%. The increase from Formulation XXX to Formulation XVI was 4.2%.

TABLE 14
Xerosol Comparison at Varying Itraconazole:PS80 Ratio

| | | | | | | | Dry |
|---|---|---|---|---|---|---|---|
| | | | | | | | Powder |
| | Itraconazole | | Sodium | | | Emitted | Inhaler |
| | Load | Itraconazole:PS | Sulfate:Leucine | MMAD | FPD < 5 μm | Dose | Retention |
| Formulation | (wt %) | 80 ratio | Ratio | (μm) | (% ND) | (% ND) | (% ND) |
| XXVIII | 50.0 | 10:1 | 3.5:1 | 3.43 | 47.6 | 72.2 | 20.6 |
| XXIX | 80.0 | 10:1 | 3.5:1 | 3.47 | 42.4 | 65.2 | 27.9 |
| XXX | 80.0 | 10:1 | 1:3.5 | 3.23 | 50.6 | 72.6 | 18.7 |
| XI | 50.0 | 12:1 | 3.5:1 | 3.12 | 53.6 | 75.3 | 16.8 |
| XIV | 80.0 | 12:1 | 3.5:1 | 3.18 | 48.8 | 68.3 | 28.6 |
| XVI | 80.0 | 12:1 | 1:3.5 | 3.31 | 54.8 | 77.8 | 13.8 |

1. A dry powder comprising homogenous respirable dry particles that comprise 1) itraconazole in crystalline particulate form, 2) polysorbate 80, and 3) one or more excipients, wherein the ratio of itraconazole to polysorbate 80 (wt:wt) in the feedstock solution is greater than 10:1, with the proviso that the dry powder formulation does not comprise: 20% Itraconazole, 39% sodium sulfate, 39% mannitol, and 2% polysorbate 80; 50% Itraconazole, 22.5% sodium sulfate, 22.5% mannitol, and 5% polysorbate 80; 20% Itraconazole, 62.4% sodium chloride, 15.6% leucine, and 2% polysorbate 80; 50% Itraconazole, 36% sodium sulfate, 9% leucine, and 5% polysorbate 80; 20% Itraconazole, 66.3% magnesium lactate, 11.7% leucine, and 2% polysorbate 80; 50% Itraconazole, 38.25% magnesium lactate, 6.75% leucine, and 5% polysorbate 80; 50% Itraconazole, 35% sodium sulfate, 10% leucine, and 5% polysorbate 80; 50% Itraconazole, 35% sodium sulfate, 10% leucine, and less than 5% polysorbate 80; 50% Itraconazole, 35% sodium sulfate, 13.75% leucine, and 1.25% polysorbate 80; 50% Itraconazole, 37% sodium sulfate, 8% leucine, and 5% polysorbate 80; 60% Itraconazole, 26% sodium sulfate, 8% leucine, and 6% polysorbate 80; 70% Itraconazole, 15% sodium, 8% leucine, and 7% polysorbate 80; 75% Itraconazole, 9.5% sodium sulfate, 8% leucine, and 7.5% polysorbate 80; 80% Itraconazole, 4% sodium sulfate, 8% leucine, and 8% polysorbate 80; 80% Itraconazole, 10% sodium sulfate, 2% leucine, and 8% polysorbate 80; 80% Itraconazole, 11% sodium sulfate, 1% leucine, and 8% polysorbate 80; or 80% Itraconazole, 11% sodium sulfate, 1% leucine, and 8% polysorbate 80.

2. The dry powder of claim 1, comprising one or more sub-particles of crystalline itraconazole, wherein the sub-particle is about 50 nm to about 5,000 nm (Dv50).

3. The dry powder of claim 1, wherein the sub-particle is about 50 nm to about 800 nm (Dv50).

4-6. (canceled)

7. The dry powder of claim 2, wherein the sub-particle is about 50 nm to about 200 nm (Dv50).

8. (canceled)

9. The dry powder of claim 1, wherein the itraconazole is present in an amount of about 1% to about 95% by weight.

10. The dry powder of claim 1, wherein the itraconazole is present in an amount of about 40% to about 90% by weight.

11-15. (canceled)

16. The dry powder of claim 1, wherein the ratio of itraconazole:polysorbate 80 (wt:wt) is from about 11.5:1 to 14:1.

17. The dry powder of claim 1, wherein the ratio of itraconazole:polysorbate 80 (wt:wt) is greater than or equal to 12:1, or about 12:1, or about 15:1 to about 19.5:1.

18. (canceled)

19. The dry powder of claim 1, wherein the polysorbate 80 is present in an amount of less than 10% by weight.

20-22. (canceled)

23. The dry powder of claim 1, wherein the one or more excipients are present in an amount of about 3% to about 99% by weight or about 5% to about 50% by weight.

24-25. (canceled)

26. The dry powder of claim 1, wherein the one or more excipients comprises a monovalent metal cation salt, a divalent metal cation salt, an amino acid, a sugar alcohol, or combinations thereof.

27. (canceled)

28. The dry powder of claim 26, wherein the monovalent metal cation salt is selected from the group consisting of sodium chloride and sodium sulfate, and the amino acid is leucine.

29. The dry powder of claim 26, wherein the monovalent metal cation salt is sodium chloride and the amino acid is leucine.

30. The dry powder of claim 26, wherein the monovalent metal cation salt is sodium sulfate and the amino acid is leucine.

31. The dry powder of claim 26, wherein the one or more excipients comprises a magnesium salt and an amino acid.

32-35. (canceled)

36. The dry powder of claim 1, wherein the respirable dry particles have a volume median geometric diameter (VMGD) about 10 microns or less.

37-38. (canceled)

39. The dry powder of claim 1, wherein the respirable dry particles have a tap density of between 0.2 g/cc and 1.0 g/cc.

40. (canceled)

41. The dry powder of claim 1, wherein the dry powder has an MMAD of between about 1 micron and about 5 microns.

42. The dry powder of claim 1, wherein the dry particles have a 1 bar/4 bar dispersibility ratio (1/4 bar) of less than about 1.5 as measured by laser diffraction.

43. The dry powder of claim 1, wherein the dry particles have a 0.5 bar/4 bar dispersibility ratio (0.5/4 bar) of about 1.5 or less as measured by laser diffraction.

44. The dry powder of claim 1, wherein the dry powder has a FPF of the total dose less than 5 microns of about 25% or more.

45. The dry powder of claim 1, wherein the dry powder is delivered to a patient with a capsule-based passive dry powder inhaler.

46. The dry powder of claim 1, wherein the respirable dry particles have a capsule emitted powder mass of at least 80% when emitted from a passive dry powder inhaler that has a resistance of about 0.036 sqrt(kPa)/liters per minute under the following conditions; an inhalation flow rate of 30 LPM for a period of 3 seconds using a size 3 capsule that contains a total mass of 10 mg, said total mass consisting of the respirable dry particles, and wherein the volume median geometric diameter of the respirable dry particles emitted from the inhaler as measured by laser diffraction is 5 microns or less.

47. A liquid formulation that comprises 1) itraconazole in crystalline particulate form, 2) polysorbate 80, and 3) one or more excipients, wherein the ratio of itraconazole to polysorbate 80 (wt:wt) in the feedstock solution is greater than 10:1.

48. The liquid formulation of claim 47, wherein the itraconazole in crystalline particulate form is suspended in a propellant selected from the group consisting of HFA propellant and CFC propellant.

49. The liquid formulation of claim 47, further comprising a surfactant.

50. A method for treating a fungal infection comprising administering to the respiratory tract of a patient in need thereof an effective amount of a dry powder of claim 1.

51. A method for treating a fungal infection in a patient with cystic fibrosis comprising administering to the respiratory tract of the cystic fibrosis patient an effective amount of a dry powder of claim 1.

52. A method for treating a fungal infection in a patient with asthma comprising administering to the respiratory tract of the asthma patient an effective amount of a dry powder of claim 1.

53. A method for treating aspergillosis comprising administering to the respiratory tract of a patient in need thereof an effective amount of a dry powder of claim 1.

54. A method for treating allergic bronchopulmonary aspergillosis (ABPA) comprising administering to the respiratory tract a patient in need thereof an effective amount of a dry powder of claim 1.

55. A method for treating or reducing the incidence or severity of an acute exacerbation of a respiratory disease comprising administering to the respiratory tract of a patient in need thereof an effective amount of a dry powder of claim 1, wherein the acute exacerbation is a fungal infection.

56. A method for treating a fungal infection in an immunocompromised patient comprising administering to the respiratory tract of the immunocompromised patient an effective amount of a dry powder of claim 1.

57-63. (canceled)

64. A dry powder of claim 1 produced by a process comprising the steps of: spray drying a surfactant-stabilized suspension with optional excipients, wherein dry particles that are compositionally homogeneous are produced.
